(19) United States
(12) Patent Application Publication
(10) Pub. No.: US 2005/0202550 A1
(43) Pub. Date: Sep. 15, 2005

(54) Crystal structure of 3', 5'-cyclic nucleotide phosphodiesterase (PDE10A) and uses thereof

(75) Inventor: Jayvardhan Pandit, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/045,860

(22) Filed: Jan. 28, 2005

Related U.S. Application Data
(60) Provisional application No. 60/552,761, filed on Mar. 12, 2004.

(51) Int. Cl. G06F 19/00; G01N 33/48; G01N 33/50; C12N 9/16

(52) U.S. Cl. 435/196; 702/19

(57) ABSTRACT

Crystal structure of phosphodiesterase 10A (PDE10A), and the 3-D atomic coordinates of the PDE10A binding domain, as described and used for the identification of ligands, including PDE10A inhibitors, used to treat various psychological disorders.

### FIELD OF THE INVENTION

The present invention relates to crystalline compositions of mammalian 3′, 5′-Cyclic Nucleotide Phosphodiesterase (PDE10A), methods of preparing said compositions, methods of determining the 3-D X-ray atomic coordinates of said composition, methods of identifying ligands of PDE10A using structure based drug design, the use of the 3-D crystal structure to design, modify and assess the activity of potential inhibitors, and to the use of such inhibitors for example, as psychotherapeutics.

### BACKGROUND OF THE INVENTION

Cyclic nucleotide second messengers (cAMP and cGMP) play a central role in signal transduction and regulation of physiologic responses. Their intracellular levels are controlled by the complex superfamily of cyclic nucleotide phosphodiesterase (PDE) enzymes. The PDE superfamily is comprised of metallophosphohydrolases (e.g., Mg²⁺, and Zn²⁺) that specifically cleave the 3′,5′-cyclic phosphate moiety of cAMP and/or cGMP to produce the corresponding 5′-nucleotide. The sensitivity of physiological processes to cAMP/cGMP signals requires that their levels be precisely maintained within a relatively narrow range in order to provide for optimal responsiveness in a cell. Cyclic nucleotide PDEs provide the major pathway for eliminating the cyclic nucleotide signal for the cell. PDEs are critical determinants for modulation of cellular levels of cAMP and/or cGMP by many stimuli.

Members of the PDE superfamily differ in their tissue distributions, physicochemical properties, substrate and inhibitor specificities and regulatory mechanisms. Based on differences in primary structure of known PDEs, they have been subdivided into two major classes, class I and class II. To date, no mammalian PDE has been included in class II. Class I contains the largest number of PDEs and includes all known mammalian PDEs. Each class I PDE contains a conserved segment of ˜250-300 amino acids in the carboxyl-terminal portion of the proteins, and this segment has been demonstrated to include the catalytic site of these enzymes. All known class I PDEs are contained within cells and vary in subcellular distribution, with some being primarily associated with the particulate fraction of the cytoplasmic fraction of the cell, others being evenly distributed in both compartments.

PDEs from mammalian tissues have been subdivided into 11 families that are derived from separate gene families. The families are named PDE1, PDE2, PDE3 . . . to PDE11. Within each family, there may be isoenzymes such as PDE1A, PDE1B and PDE1C, and PDE10A1 and PDE10A2. PDEs within a given family may differ significantly but the members of each family are functionally related to each other through similarities in amino acid sequences, specificities and affinities for cGMP (PDE5, PDE6, and PDE9) or cAMP (PDE4, PDE7, and PDE8) or accommodation of both (PDE1, PDE2, PDE3, PDE10, and PDE11), inhibitor specificities, and regulatory mechanisms.

Comparison of the amino acid sequences of PDEs suggests that all PDEs may be chimeric multidomain proteins possessing distinct domains that provide for catalysis and a number of regulatory functions. The amino acid sequences of all mammalian PDEs identified to date include a highly conserved region of approximately 270 amino acids located in the carboxy terminal half of the proteins. (Charbonneau, et al., Proc. Natl. Acad., Sci. (USA) 83:9308-9312 (1986)). The conserved domain includes the catalytic site for cAMP and/or cGMP hydrolysis and two putative metal (presumably zinc) binding sites as well as family specific determinants. (Beavo, Physiol. Rev. 75: 725-748 (1995); Francis, et al., J. Biol. Chem. 269:22477-22480 (1994)). The amino terminal region of the various PDEs are highly variable and include other family specific determinants such as: (i) calmodulin binding sites (PDE1); (ii) non-catalytic cGMP binding sites (PDE2, PDE5, PDE6); (iii) membrane targeting sites (PDE4); (iv) hydrophobic membrane association sites (PDE3); and (v) phosphorylation sites for either the calmoduline-dependent kinase (II) (PDE1), the cAMP-dependent kinase (PDE1, PDE3, PDE4), or the cGMP dependent kinase (PDE5) (Beavo, Physiol. Rev. 75:725-748 (1995); Manganiello, et al., Arch. Biochem. Acta 322: 1-13 (1995); Conti, et al., Physiol. Rev. 75:723-748 (1995); WO 99/42596).

While all known mammalian PDEs are either dimeric or oligomeric, the functional importance of this quaternary structure is not known, and experiments further indicate that in some PDEs the components required for catalyzing hydrolysis of the phosphodiester bond in cAMP or cGMP are contained in a single catalytic domain and that the interactions between two catalytic domains within a dimer or between the catalytic domain and the regulatory domain are not required for this process. (See Francis, S. H. et al., Prog. Nuc. Acid Res Molec. Biol., 65: 1-52, 2001).

PDE10 is identified as a unique family within the PDE superfamily based on primary amino acid sequence and distinct enzymatic activity. Homology screening of EST databases has revealed mouse PDE10A as the first member of the PDE10 family of the PDE10 family of phosphodiesterases. (Fujishige et al., J. Biol. Chem. 274: 18438-18445, 1999; Lougheny, K. et al., Gene 234-109-117, 1999) The murine homologue has also been cloned (Solderling, S. et al., Eur. J. Biochem. 266: 1118-1127, 1999). The mouse PDE10A1 is a 779 amino acid protein that hydrolyzes both cAMP and cGMP to AMP and GMP, respectively. The affinity of the PDE10 for cAMP (Km=0.05 uM) is higher than for cGMP (Km=3 uM). However, the approximately 5-fold greater Vmax for cGMP over cAMP has lead to the suggestion that PDE10 is a cAMP-inhibited cGMPase. (Fujishige et al., J. Biol. Chem. 274, 18438-18445, 1999; EP 1250923). The human gene encoding for PDE10 has been cloned and found to span over 200 kb with 24 exons. (Fujishigi, K. et al., Eur. J. Biochem., 267, pages 5943-5951 (2000)).

PDE10 is uniquely localized in mammals relative to other PDE families. It is reported, that mRNA for PDE10 is highly expressed only in testis and brain. (Fujishige et al., J. Biol. Chem. 274, 18438-18445, 1999; Soderling et al., Proc. Natl. Acad. Sci. (USA) 96, 7071-7078, 1999; Lougheny, K. et al., Gene 234-109-117, 1999). These initial studies indicated that within the brain PDE10 expression is highest in the striatum (caudate and putamen), n. accubens, and olfactory tubercle. More recently, a detailed analysis has been made of the expression pattern of PDE10 mRNA in rodent brain. (Seeger et al., Abst. Soc. Neurosci., 26:345, 10, 2000).

Selective inhibition of PDE10 has been investigated for the treatment of various diseases of the central nervous system. EP 1250923 discloses several specific inhibitors of PDE10 with antipsychotic properties useful in the treatment of disorders including, multiple variants of schizophrenia, anxiety disorders, movement disorders selected from Huntington's disease, Parkinson's disease and dyskinesia, alcohol and drug addictions, cognitive deficiencies, and mood disorders.

Several methods have been used in the past and continue to be used to discover selective inhibitors of biomolecular targets such as PDE10. The various approaches include ligand-directed drug discovery (LDD), quantitative structure activity relationship (QSAR) analyses; and comparative molecular field analysis (CoMFA). CoMFA is a particular type of QSAR method that uses statistical correlation techniques for the analysis of the quantitative relationship between the biological activity of a set of compounds with a specified alignment, and their three-dimensional electronic and steric properties. Other properties such as hydrophobicity and hydrogen bonding can also be incorporated into the analysis.

An invaluable component of these drug discovery approaches is structure based design, which is a design strategy for new chemical entities, or optimization of lead compounds identified by other methods using the three-dimensional (3D) structure of the biological macromolecule target obtained by for example, X-ray or nuclear magnetic resonance NMR studies, or from homology models. Analyzing 3-D structures of proteins provides crucial insights into the behavior and mechanics of drug binding and biological activity. Coupled with computational techniques including modeling and simulation, the study of biomolecular interactions provides details of events that may be difficult to investigate experimentally in the laboratory, and can help reveal topological features important for determining molecular recognition. As those skilled in the art will recognize, this information can, in turn, be used for predicting ligand-receptor complex formation, and for designing ligands and protein mutations that produce desired ligand receptor interactions.

Regulation of PDEs is important for controlling myriad physiological functions, including the visual response, smooth muscle relaxation, platelet aggregation, fluid homeostasis, immune responses, and cardiac contractility. PDEs are critically involved in feedback control of cellular cAMP and cGMP levels. The PDE superfamily continues to be a major target for pharmacological intervention in a number of medically important maladies including cardiovascular diseases, asthma, depression, and male impotence. For example, PDE5, found in varying concentrations in a number of tissues, has been recognized in recent years as an important therapeutic agent. (See U.K. Patent Application 0126417.5, filed Nov. 2, 2001). To that end the quest for specific and potent PDE inhibitors for use in physiological studies and therapeutic settings continues. Thus, obtaining, three-dimensional (3D) structures of PDEs, such as PDE10A, obtained by for example, X-ray or nuclear magnetic resonance NMR studies, or from homology models, and analyzing the structures using computational methods facilitates such discovery efforts.

### SUMMARY OF THE INVENTION

The present invention provides crystalline compositions of PDE10A, and specifically of the catalytic region of PDE10A. The invention further provides methods of preparing said compositions, methods of determining the 3-D X-ray atomic coordinates of said crystalline compositions, methods of using the atomic coordinates in conjunction with computational methods to identify binding site(s), methods to elucidate the 3-D structure of homologues of PDE10A, and methods to identify ligands which interact with the binding site(s) to agonize or antagonize the biological activity of PDE10A, methods for identifying inhibitors of PDE10A, pharmaceutical compositions of inhibitors, and methods of treatment of psychotherapeutic disorders using said pharmaceutical compositions.

In a preferred embodiment the invention provides crystalline compositions of the catalytic region of PDE10A.

One aspect of the present invention provides methods for crystallizing a PDE10A polypeptide ligand complex comprising a polypeptide. Preferably the methods for crystallizing a PDE10A polypeptide ligand complex comprising an amino acid sequence spanning the amino acids 442 to 774 listed in SEQ ID NO:1 comprising: (a) preparing solutions of the polypeptide, ligand and precipitant; (b) growing a crystal comprising molecules of the polypeptide from said mixture solution; and (c) separating said crystal from said solution. The crystallization growth can be carried out by various techniques known by those skilled in the art, such as for example, batch crystallization, liquid bridge crystallization, or dialysis crystallization. Preferably, the crystallization growth is achieved using vapor diffusion techniques.

An embodiment of the present invention provide crystalline compositions of PDE10A comprising a crystalline form of a polypeptide with an amino acid sequence spanning the amino acids Thr442 to Asp774 listed in SEQ ID NO:1, wherein the crystalline composition has a space group R3 and unit cell dimensions a=b=120.56 Å, c=82.23 Å.

In a second aspect, the present invention provides vectors useful in methods for preparing a substantially purified C-terminal catalytic domain of PDE10A comprising the polypeptide with an amino acid sequence spanning amino acids Thr442 to Asp774 listed in SEQ ID NO:1.

In a third aspect, the present invention provides methods for determining the X-ray atomic coordinates of the crystalline compositions at a 2 Å or 1.8 Å resolution.

In a fourth aspect, the present invention provides a molecule or molecular complex crystal, wherein the crystal has substantially similar atomic coordinates to the atomic coordinates listed in FIG. 1 or portions thereof, or any scalable variations thereof.

In a fifth aspect, the present invention provides a molecule or molecular complex crystal, wherein the crystal comprises a polypeptide with an amino acid sequence spanning the amino acids Thr442 to Asp774 listed in SEQ ID NO:1. A further embodiment of the invention provides a crystal comprising an amino acid sequence that is at least 98%, 95% or 90% homologous to a polypeptide with an amino acid sequence spanning the amino acids Thr442 to Asp774 listed in SEQ ID NO:1.

An even further embodiment of the invention provides a crystal comprising an amino acid sequence that is at least 98%, 95% or 90% homologous to a polypeptide with an amino acid sequence spanning the amino acids Thr442 to Asp774 listed in SEQ ID NO:1, and which has the atomic coordinates listed in FIG. 4.

In a sixth aspect, the present invention provides a molecule or molecular complex crystal, wherein the crystal comprises a polypeptide, or a portion thereof, with atomic coordinates having a root mean square deviation from the protein backbone atoms (N, Cα, C, and O) listed in FIG. 1 of less than 0.2, 0.5, 0.7, 1.0, 1.2 or 1.5 Å.

In a seventh aspect, the present invention provides a scalable, or translatable, three dimensional configuration of points derived from structural coordinates of at least a portion of a PDE10A molecule or molecular complex comprising a polypeptide with an amino acid sequence spanning the amino acids Thr442 to Asp774 listed in SEQ ID NO:1. In an embodiment of this aspect, the invention also comprises the structural coordinates of at least a portion of a molecule or a molecular complex that is structurally homologous to a PDE10A molecule or molecular complex. On a molecular scale, the configuration of points derived from a homologous molecule or molecular complex have a root mean square deviation of less than about 0.2, 0.5, 0.7, 1.0, 1.2 or 1.5 Å from the structural coordinates provided in FIG. 4.

In an eight aspect, the present invention provides a computer for producing a three-dimensional representation of:

a. a molecule or molecular complex comprising a polypeptide with an amino acid sequence spanning amino acids Thr442 to Asp774 listed in SEQ ID NO:1, or a homologue, or a variant thereof;

b. a molecule or molecular complex, wherein the atoms of the molecule or molecular complex are represented by atomic coordinates that are substantially similar to, or are subsets of the atomic coordinates listed in FIG. 4;

c. a molecule or molecular complex, wherein the molecule or molecular complex comprises atomic coordinates having a root mean square deviation of less than 0.2, 0.5, 0.7, 1.0, 1.2 or even 1.5 Å from the atomic coordinates for the carbon backbone atoms listed in FIG. 1; or

d. a molecule or molecular complex, wherein the molecule or molecular complex comprises a binding pocket or site defined by the structure coordinates that are substantially similar to the atomic coordinates listed in FIG. 4, or a subset thereof, or more preferably the structural coordinates in FIG. 4 corresponding to one or more PDE10A amino acids, or conservative replacements thereof, in SEQ ID NO: 1 selected from Leu625, Phe629, Val668, Phe686, Met703, Gln716 and Phe719,

wherein said computer comprises:

(i) a computer-readable data storage medium comprising a data storage medium encoded with computer-readable data, wherein said data comprises the structure coordinates of FIG. 4, or portions thereof, or substantially similar coordinates thereof;

(ii) a working memory for storing instructions for processing said computer-readable data;

(iii) a central-processing unit coupled to said working memory and to said computer-readable data storage medium for processing said computer-machine readable data into said three-dimensional representation; and

(iv) a display coupled to said central-processing unit for displaying said representation.

The computer configured according to this aspect of the invention can be used to design and identify potential ligands or inhibitors of PDE10A by, for example commercially available molecular modeling software in conjunction with structure-based drug design as provided herein.

In a ninth aspect, the present invention provides methods involving molecular replacement to obtain structural information about a molecule or molecular complex of unknown structure. In one embodiment, the method includes crystallizing the molecule or molecular complex, generating an x-ray diffraction pattern from the crystallized molecule or molecular complex, and applying at least a portion of the structure coordinates set forth in FIG. 4 to the x-ray diffraction pattern to generate a three-dimensional electron density map of at least a portion of the molecule or molecular complex.

In another embodiment, the present invention provides methods for generating 3-D atomic coordinates of a protein homologue or a variant of PDE10A using the X-ray coordinates of PDE10A described in FIG. 4, comprising,

a. identifying one or more homologous polypeptide sequences to PDE10A;

b. aligning said sequences with the sequence of PDE10A which comprises a polypeptide with an amino acid sequence spanning amino acids Thr442 to Asp774 listed in SEQ ID NO:1;

c identifying structurally conserved and structurally variable regions between said homologous sequence(s) and PDE10A;

d. generating 3-D coordinates for structurally conserved residues of the said homologous sequence(s) from those of PDE10A using the coordinates listed in FIG. 4;

e. generating conformations for the loops in the structurally variable regions of said homologous sequence(s);

f. building the side-chain conformations for said homologous sequence(s); and

g. combining the 3-D coordinates of the conserved residues, loops and side-chain conformations to generate full or partial 3-D coordinates for said homologous sequences.

Embodiments of the ninth aspect provide methods, which further comprise refining and evaluating the full or partial 3-D coordinates. These methods may thus be used to generate 3-dimensional structures for proteins for which heretofore 3-dimensional atomic coordinates have not been determined. Depending on the extent of sequence homology, the newly generated structure can help to elucidate enzymatic mechanisms, or be used in conjunction with other molecular modeling techniques in structure based drug design.

In the tenth aspect, the present invention provides methods for identifying inhibitors, ligands, and the like of PDE10A by providing the coordinates of a molecule of PDE10A to a computerized modeling system; identifying chemical entities that are likely to bind to or interfere with the molecule (e.g., by screening a small molecule library); and, optionally, procuring or synthesizing and assaying the compounds or analogues derived thereof for bioactivity. In certain embodiments the present invention relates to methods for identifying potential ligands for PDE10A or homologues or variants thereof comprising:

a. displaying the three dimensional structure of PDE10A enzyme or homologue or variant thereof, or portions thereof, as defined by atomic coordinates that are substantially similar to the atomic coordinates listed in FIG. 4 on a computer display screen;

b. optionally replacing one or more the enzyme amino acid residues listed in SEQ ID NO:1, or preferably one or more amino acid residues selected from Leu625, Phe629, Val668, Phe686, Met703, Gln716 and Phe719, in said three-dimensional structure with a different naturally occurring amino acid or an unnatural amino acid to display a variant structure;

c. optionally conducting ab intio, molecular mechanics or molecular dynamics calculations on the displayed three dimensional structure to generate a modified structure;

d. employing said three-dimensional structure, variant structure, or modified structure to design or select said ligand;

d. synthesizing or obtaining said ligand;

e. contacting said ligand with said enzyme in the presence of one or more substrates; and

f. measuring the ability of said ligand to modulate the activity of said enzyme.

Those of skill in the art can appreciate that the information obtained by the methods for identifying inhibitors and ligands of PDE10A, as described above, can be used to iteratively refine or modify the structure of original ligand. Thus, once a ligand is found to modulate the activity of said enzyme, the structural aspects of the ligand may be modified to generate a structural analog of the ligand. This analog can then be used in the above method to identify binding ligands. One of ordinary skill in the art will know the various ways a structure may be modified.

In embodiments, preferred ligands include selective inhibitor of PDE10A.

In embodiments, the methods further comprise computationally modifying the structure of the ligand; computationally determining the fit of the modified ligand using the three-dimensional coordinates described in FIG. 4, or portions thereof; contacting said modified ligand with said enzyme, or homologue, or variant thereof in an in vitro or in vivo setting; and measuring the ability of said ligand to modulate the activity of said enzyme.

In an eleventh aspect, the present invention provides compositions and pharmaceutical preparations comprising the inhibitors or ligands designed according to any of the above methods. In one embodiment, a composition is provided that includes an inhibitor or ligand designed or identified by any of the above methods. In another embodiment, the composition is a pharmaceutical composition.

The twelfth aspect of the present invention are methods for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising administering pharmaceutical compositions, identified by structure based design using the atomic coordinates, or portions thereof, listed in FIG. 4, effective in treating the disorders or conditions.

### BRIEF DESCRIPTION OF THE DRAWINGS

FIG. 1 is an orthogonal view of the embodiment of PDE10A in ribbon representation. The compound of Formula 1 is shown in ball-and-stick representation. N- and C-termini of the polypeptide are labeled.

FIG. 2 is another orthogonal view of the embodiment of the compound of Formula 1 with PDE10A.

FIG. 3 is schematic diagram showing the interactions of the compound of Formula 1 with PDE10A.

FIG. 4 is a list of the X-ray coordinates of the PDE10A C-terminal catalytic domain crystal as described in the Examples.

### DETAILED DESCRIPTION OF THE INVENTION

The present invention relates to crystalline compositions of PDE10A, 3-D X-ray atomic coordinates of said crystalline composition, methods of preparing said compositions, methods of determining the 3-D X-ray atomic coordinates of said crystalline compositions, and methods of using said atomic coordinates in conjunction with computational methods to identify binding site(s), or identify ligands which interact with said binding site(s) to agonize or antagonize PDE10A.

For convenience, certain terms employed in the specification, examples, and appendant claims are collected here. Unless defined otherwise, all technical and scientific terms used herein have the same meaning as commonly understood by one of ordinary skill in the art to which this invention belongs.

The term “affinity” as used herein refers to the tendency of a molecule to associate with another. The affinity of a drug is its ability to bind to its biological target (receptor, enzyme, transport system, etc.) For pharmacological receptors, affinity can be thought of as the frequency with which the drug, when brought into the proximity of a receptor by diffusion, will reside at a position of minimum free energy within the force field of that receptor.

The term “agonist” as used herein refers to an endogenous substance or a drug that can interact with a receptor and initiate a physiological or a pharmacological response characteristic of that receptor (contraction, relaxation, secretion, enzyme activation, etc.)

The term “analog” as used herein refers to a drug or chemical compound whose structure is related in some way to that of another drug or chemical compound, but whose chemical and biological properties may be quite different.

The term “antagonist” as used herein refers to a drug or a compound that opposes the physiological effects of another. At the receptor level, it is a chemical entity that opposes the receptor-associated responses normally induced by another bioactive agent.

As used herein the term “binding site” refers to a specific region (or atom) in a molecular entity that is capable of entering into a stabilizing interaction with another molecular entity. In certain embodiments the term also refers to the reactive parts of a macromolecule that directly participate in its specific combination with another molecule. In other embodiments, a binding site may be comprised or defined by the three dimensional arrangement of one or more amino acid residues within a folded polypeptide. In further embodiments, the binding site further comprises prosthetic groups, water molecules or metal ions which may interact with one or more amino acid residues. Prosthetic groups, water molecules, or metal ions may be apparent from X-ray crystallographic data, or may be added to an apo protein or enzyme using in silico methods.

The term “bioactivity” refers to PDE10A activity that exhibits a biological property conventionally associated with a PDE10A agonist or antagonist, such as a property that would allow treatment of one or more of the various diseases of the central nervous system.

The term “catalytic domain” as used herein, refers to the catalytic domain of the PDE10A class of enzymes, which features a conserved segment of amino acids in the carboxy-terminal portion of the proteins, wherein this segment has been demonstrated to include the catalytic site of these enzymes. This conserved catalytic domain extends approximately from residue Thr442 to Asp774 of the full length enzyme.

“To clone” as used herein, means obtaining exact copies of a given polynucleotide molecule using recombinant DNA technology. Furthermore, “to clone into” may be meant as inserting a given first polynucleotide sequence into a second polynucleotide sequence, preferably such that a functional unit combining the functions of the first and the second polynucleotides results. For example, without limitation, a polynucleotide from which a fusion protein may be translationally provided, which fusion protein comprises amino acid sequences encoded by the first and the second polynucleotide sequences. Specifics of molecular cloning can be found in a number of commonly used laboratory protocol books such as Molecular Cloning: A Laboratory Manual, 2nd Ed., ed. by Sambrook, Fritsch and Maniatis (Cold Spring Harbor Laboratory Press: 1989).

The term “co-crystallization” as used herein is taken to mean crystallization of a preformed protein/ligand complex.

The term “complex” or “co-complex” are used interchangeably and refer to a PDE10A molecule, or a variant, or homologue of PDE10A in covalent or non-covalent association with a substrate, or ligand.

The term “contacting” as used herein applies to in silico, in vitro, or in vivo experiments.

As used herein, the terms “gene”, “recombinant gene” and “gene construct” refer to a nucleic acid comprising an open reading frame encoding a polypeptide, including both exon and (optionally) intron sequences. The term “intron” refers to a DNA sequence present in a given gene which is not translated into protein and is generally found between exons.

The term “high affinity” as used herein means strong binding affinity between molecules with a dissociation constant K_{D} of no greater than 1 mM. In a preferred case, the K_{D} is less than 100 nM, 10 nM, 1 nM, 100 pM, or even 10 pM or less. In a most preferred embodiment, the two molecules can be covalently linked (K_{D} is essentially 0).

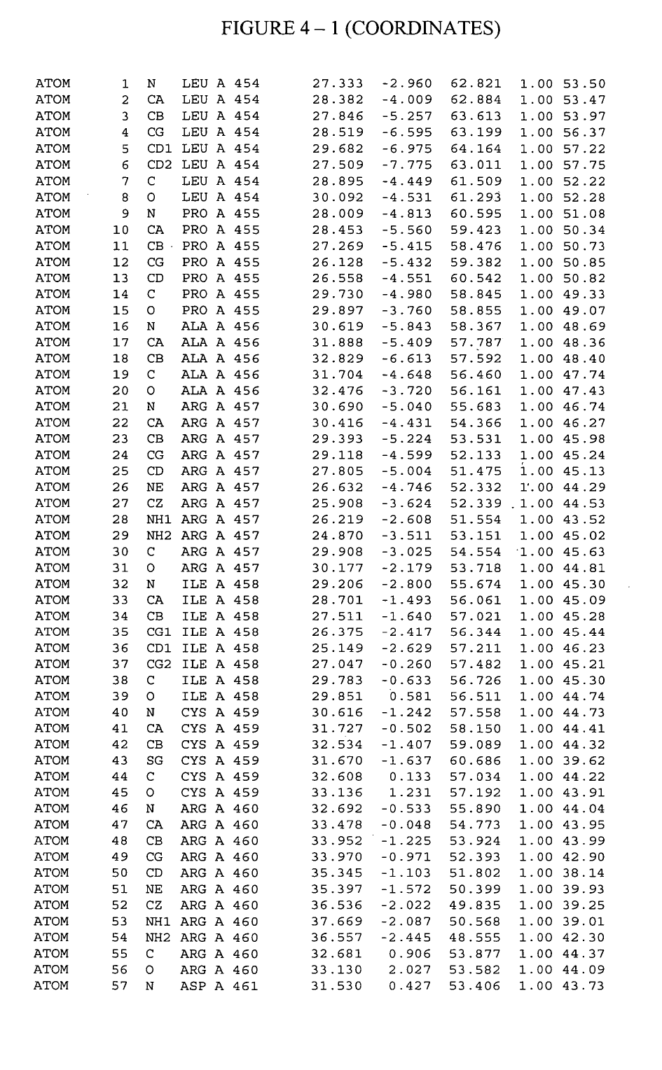

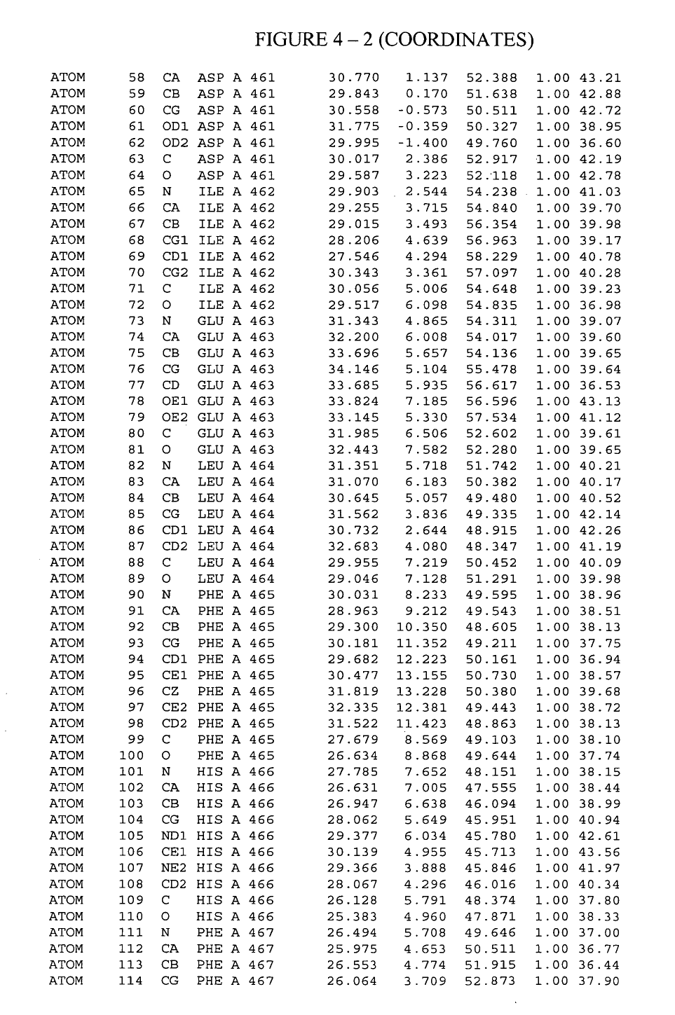

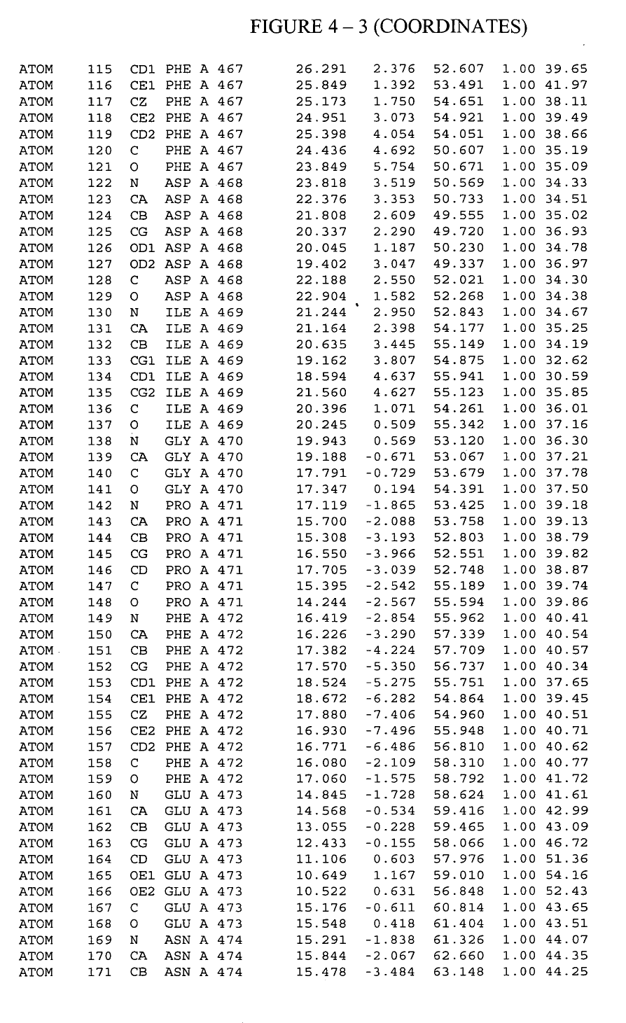

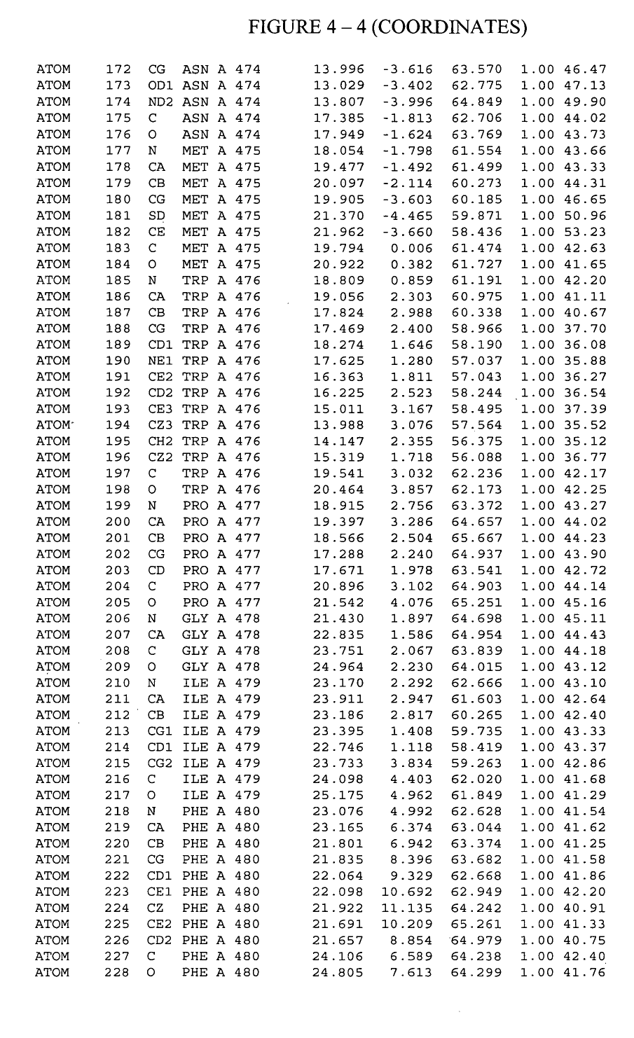

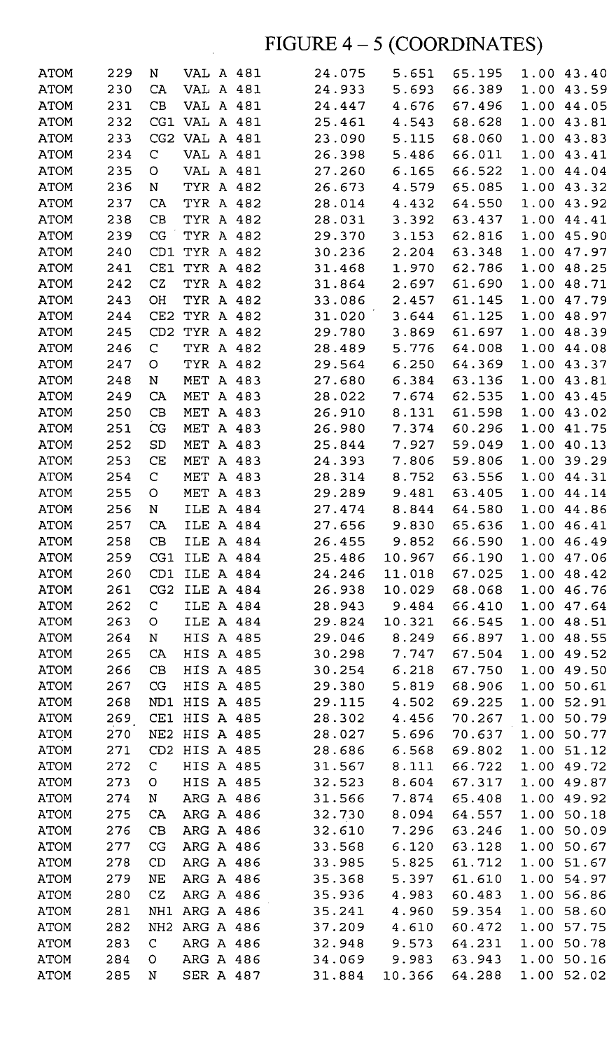

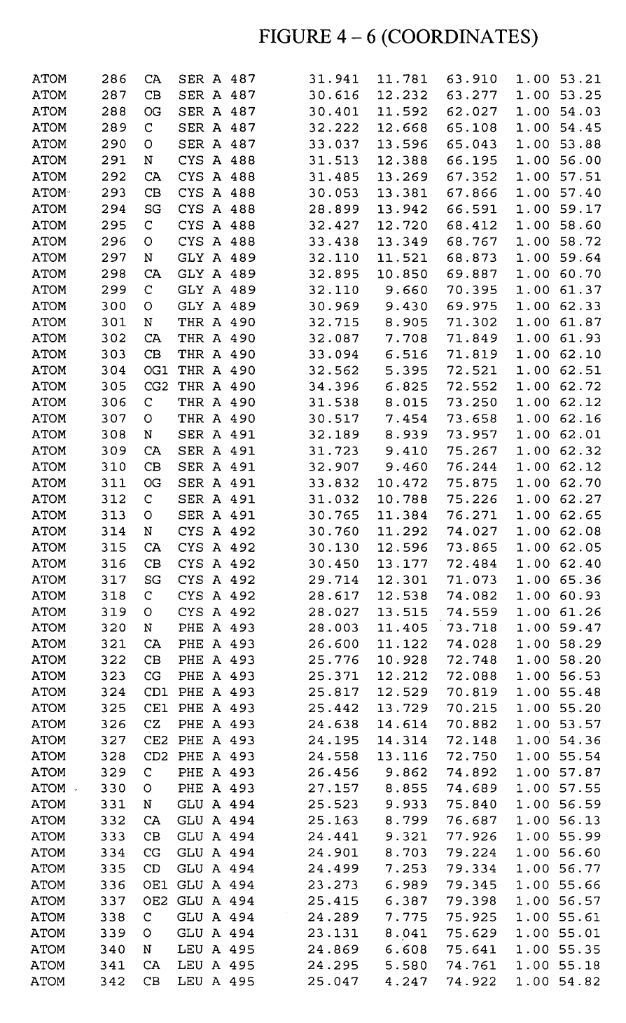

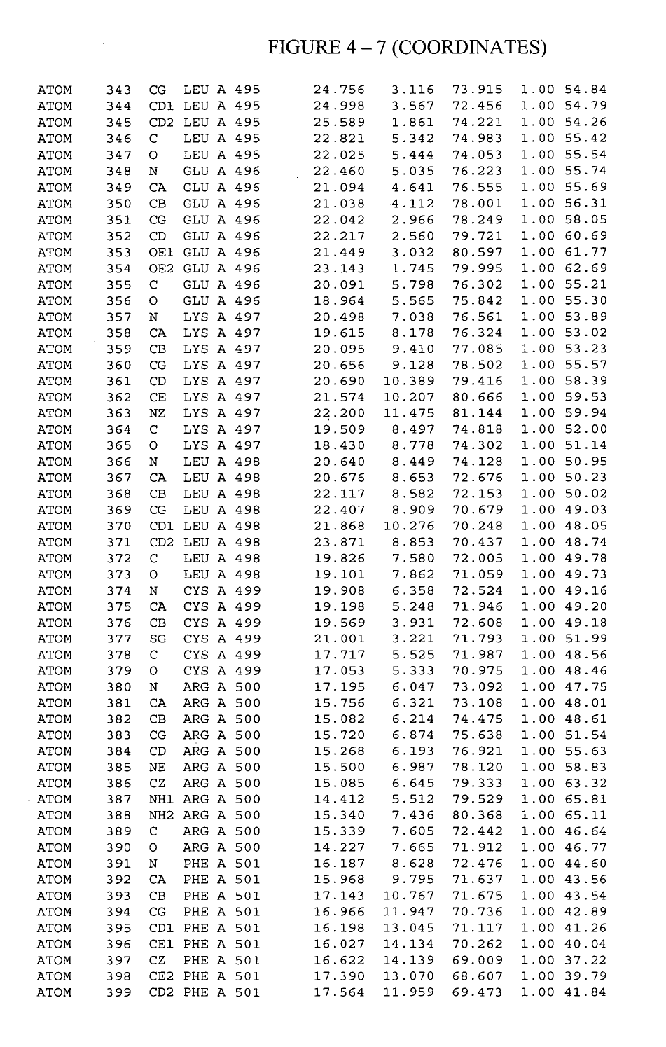

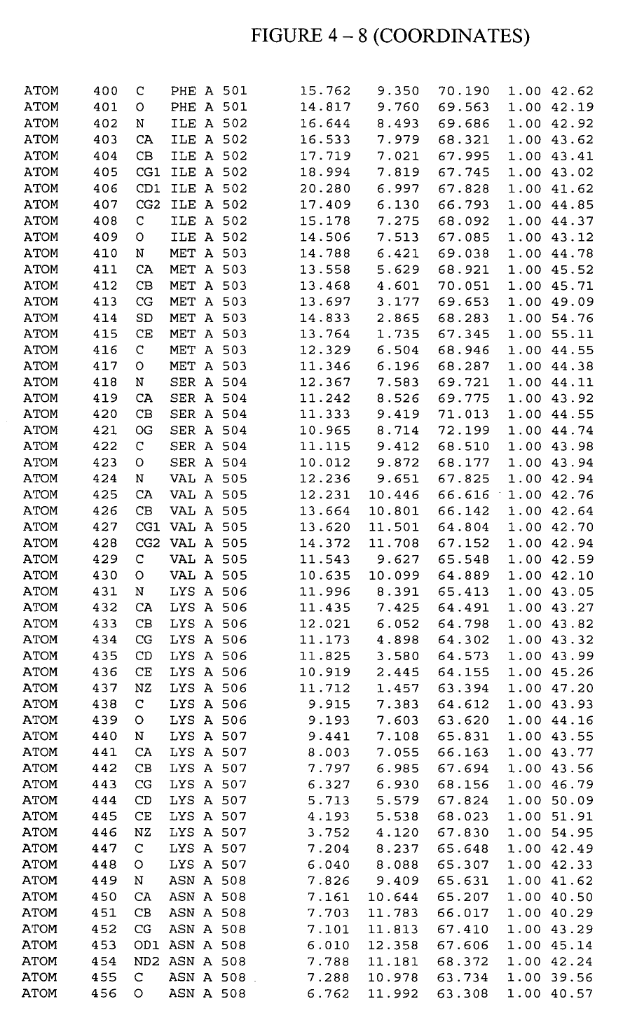

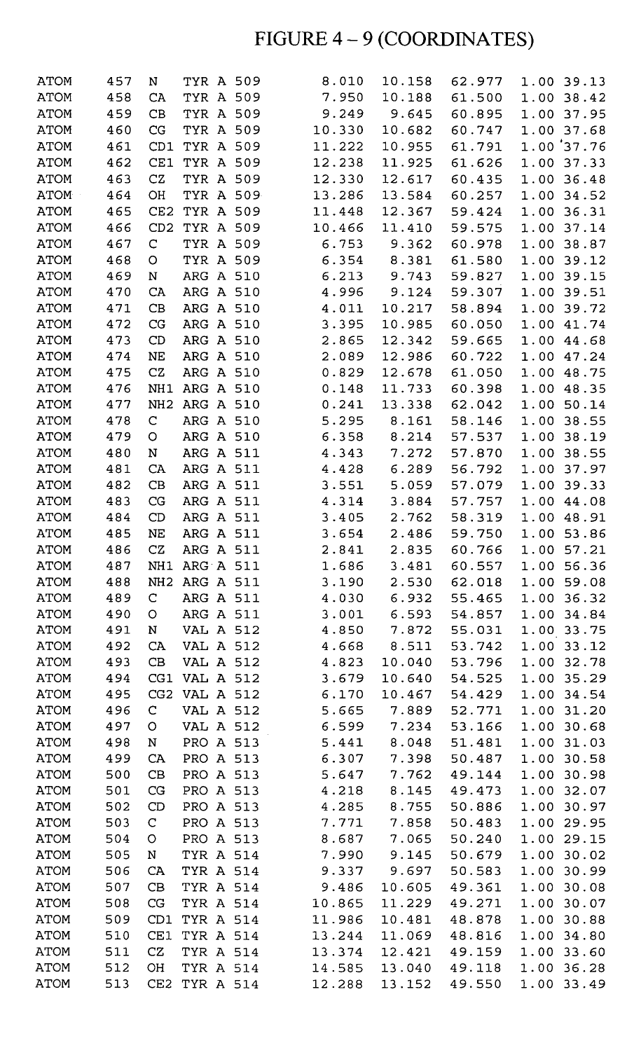

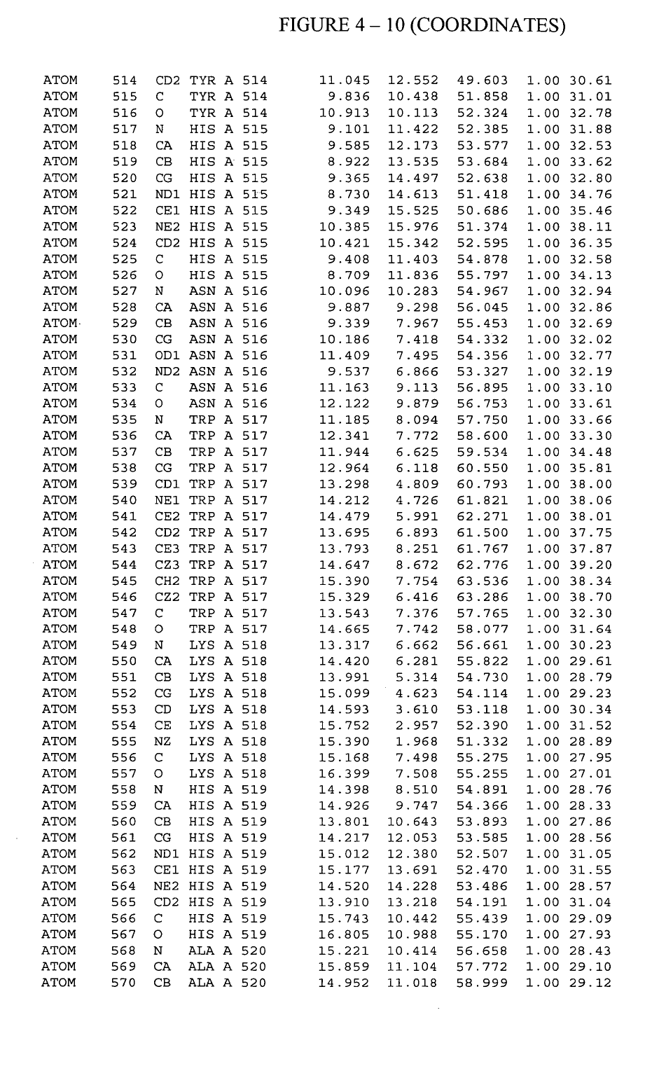

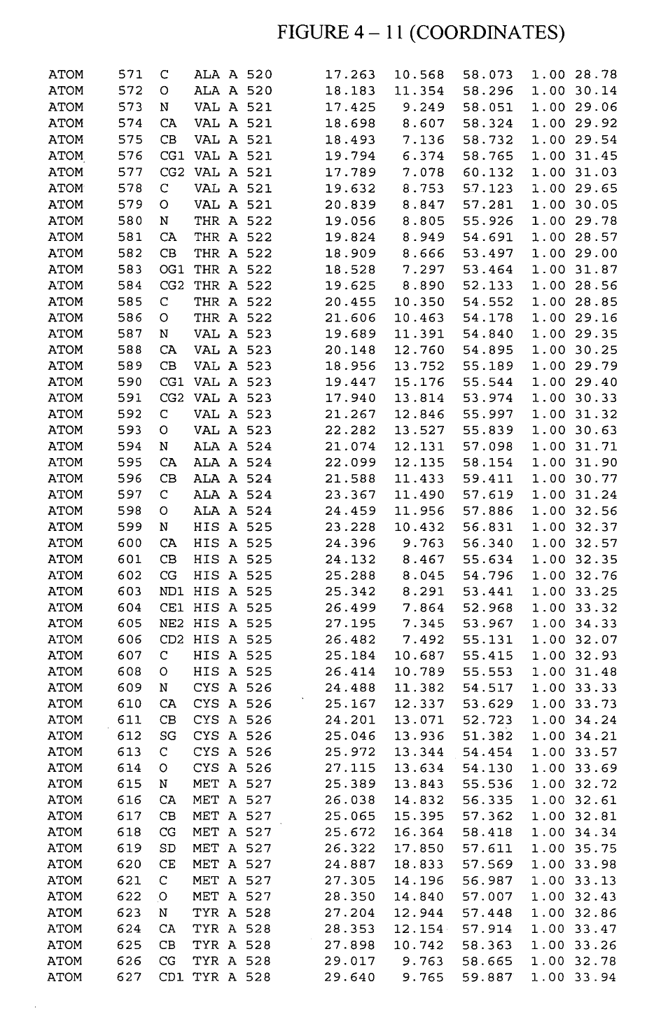

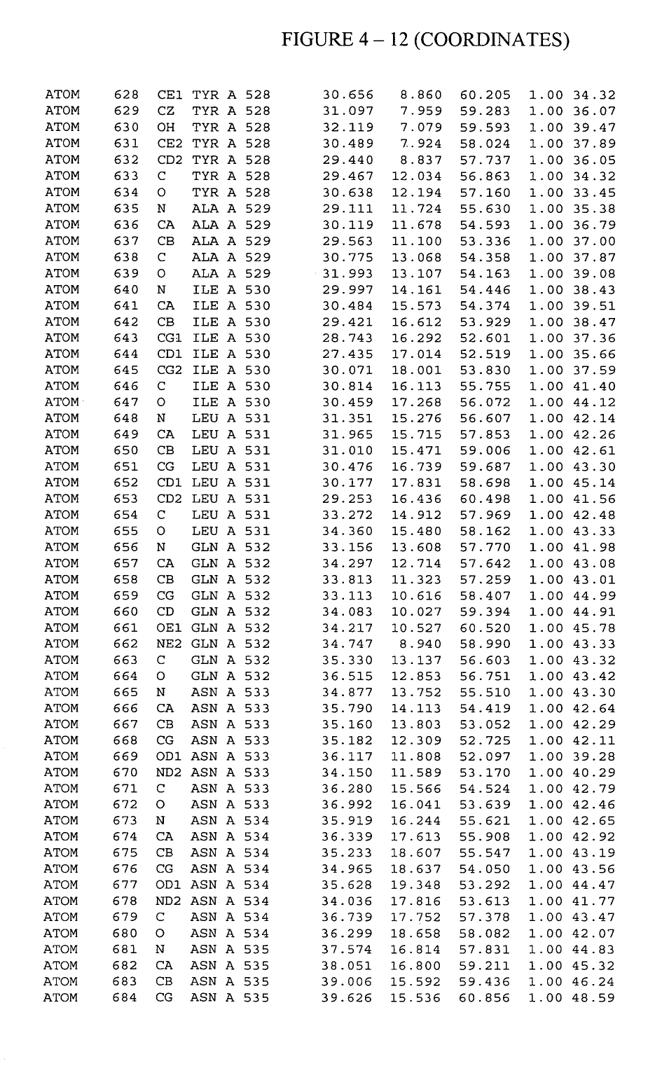

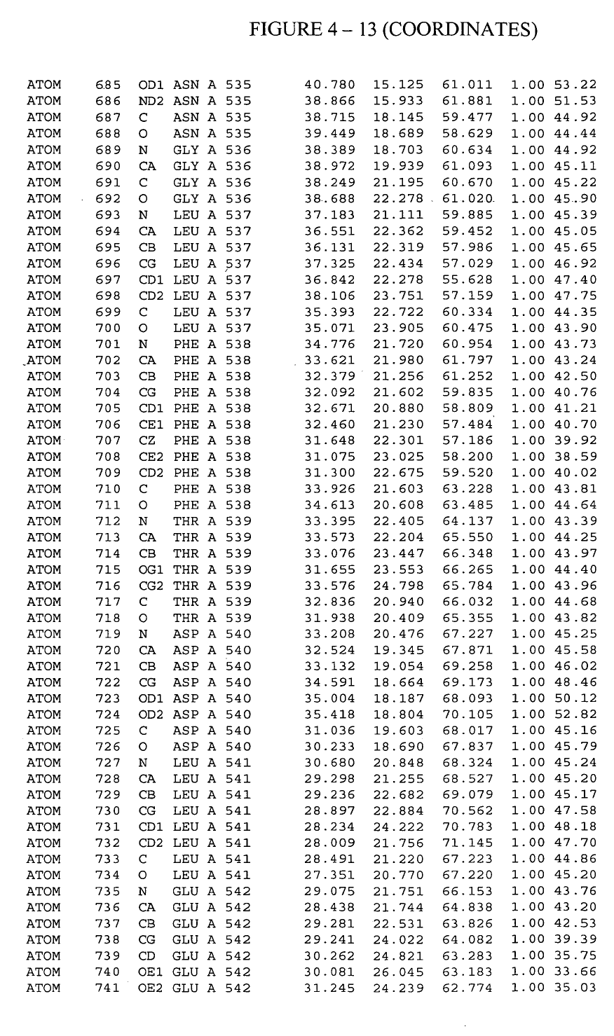

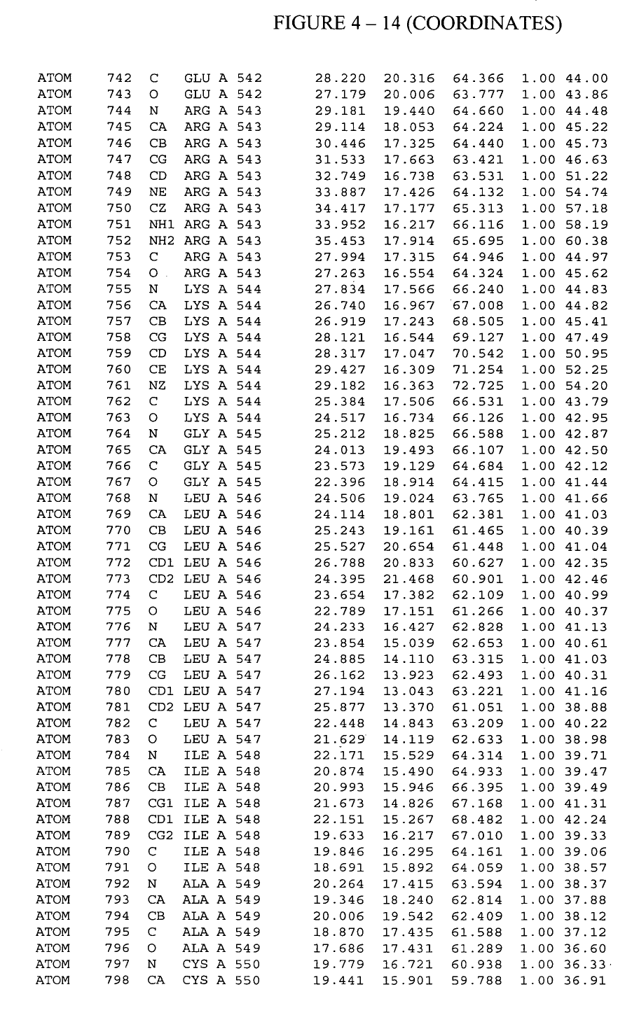

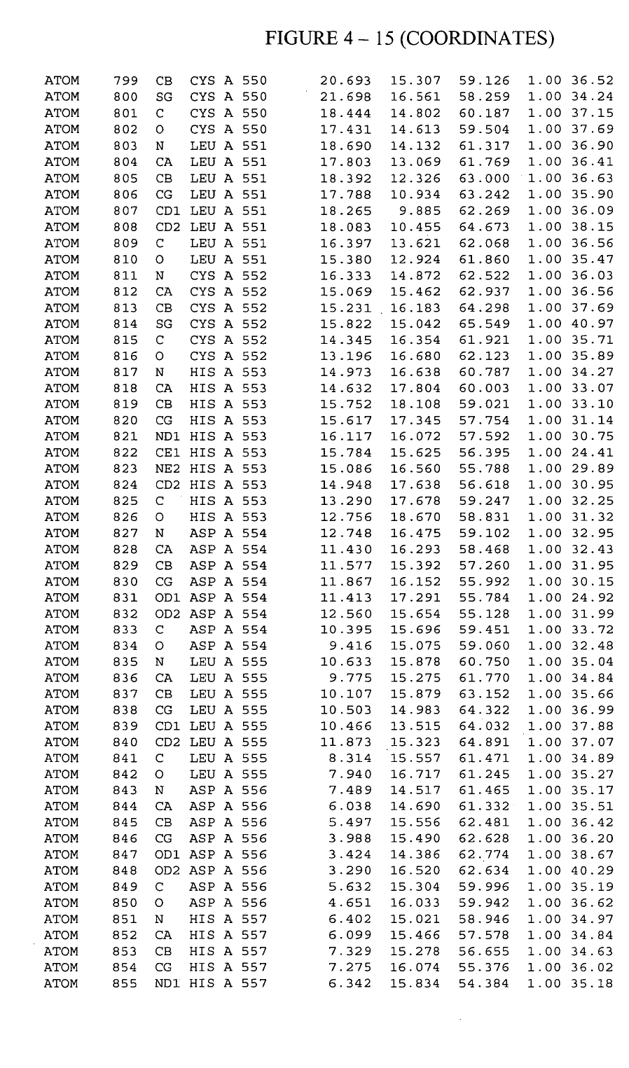

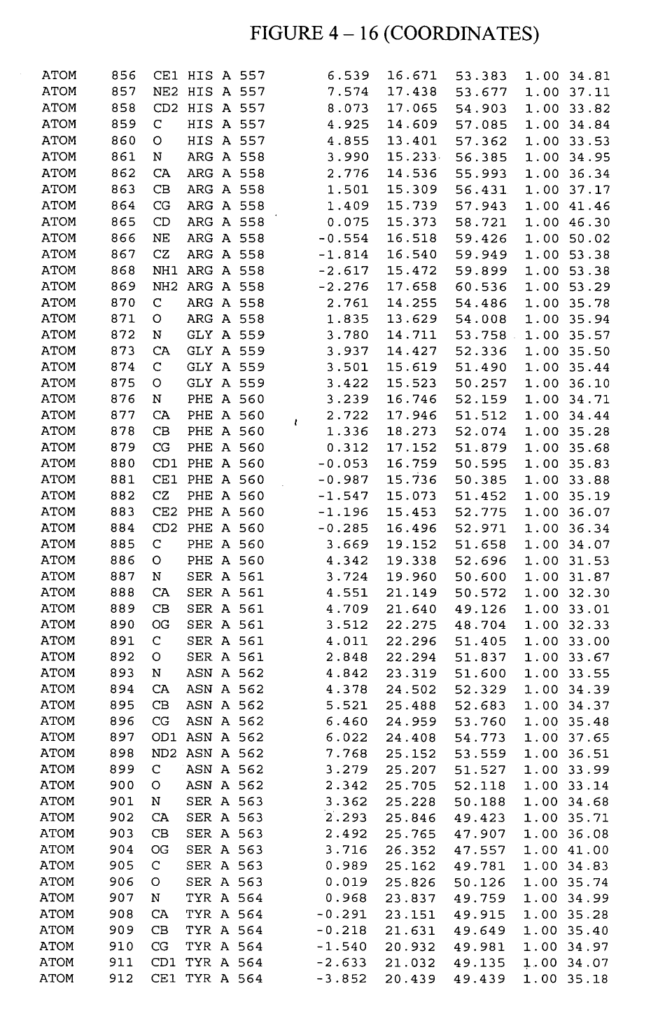

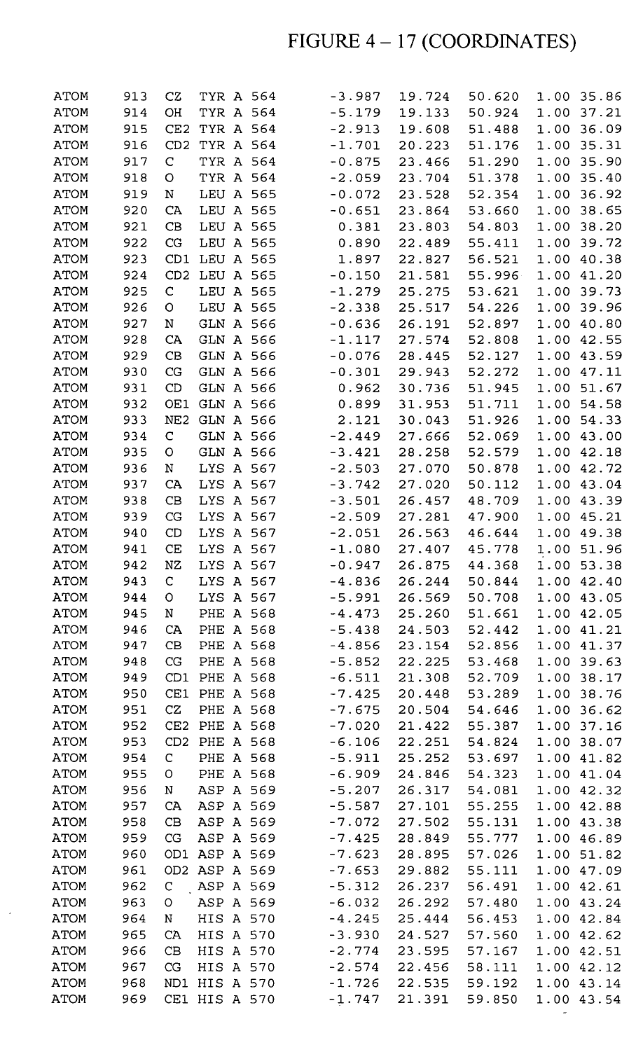

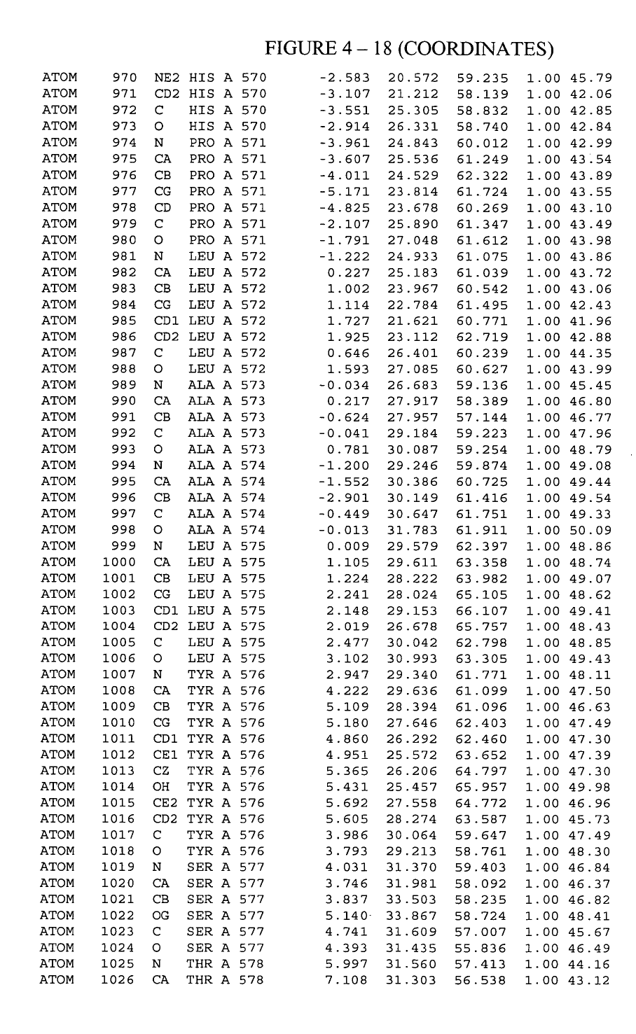

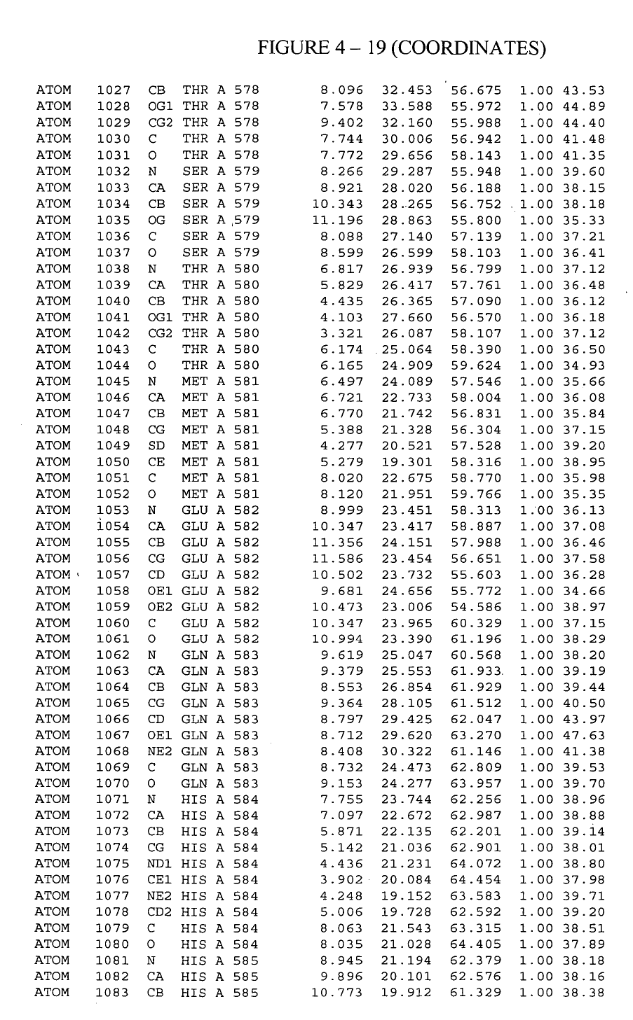

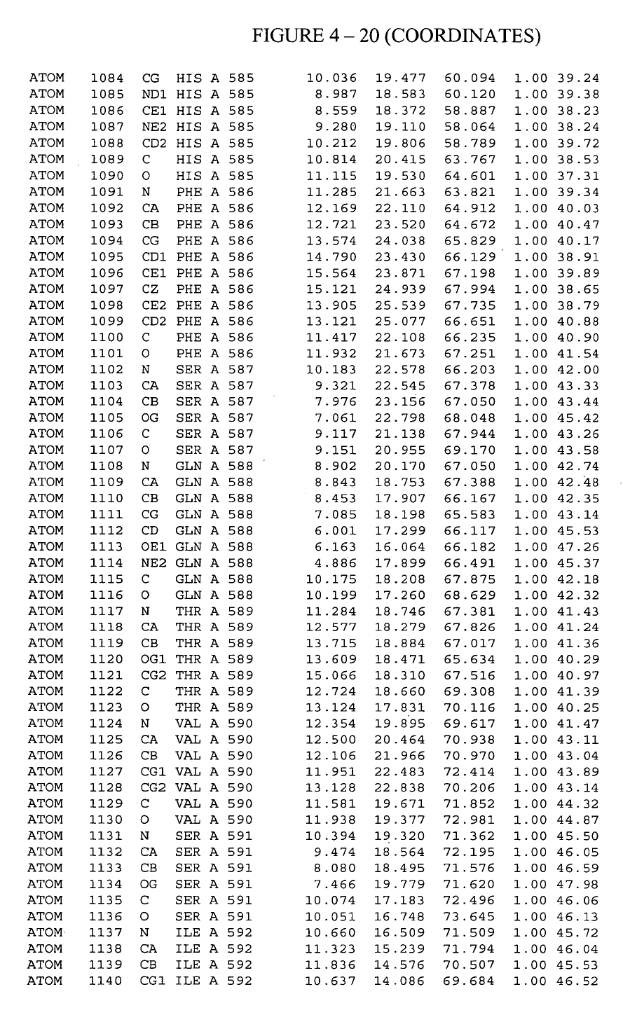

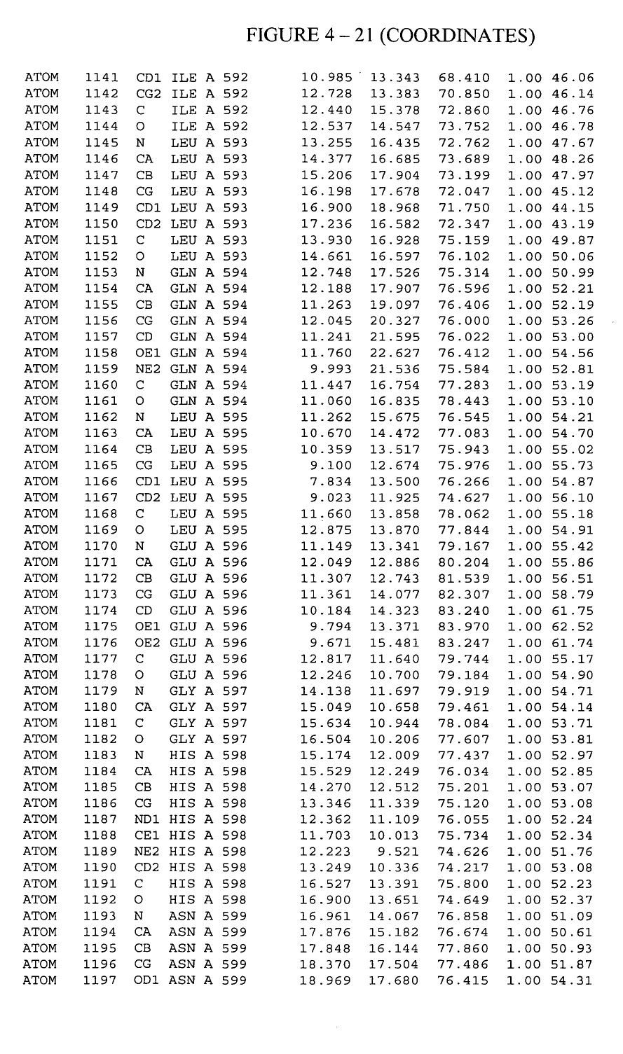

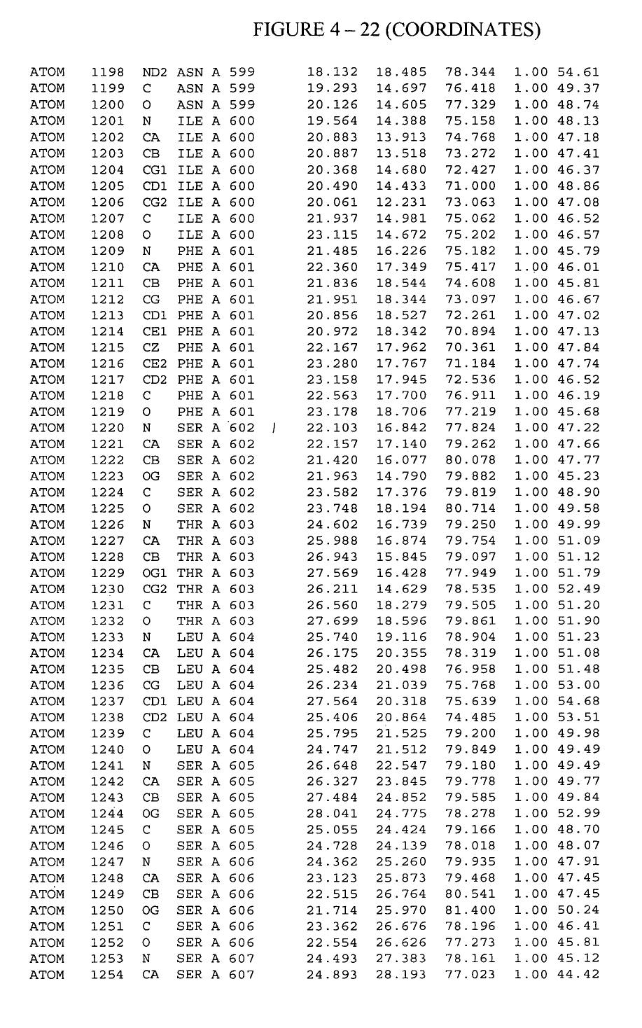

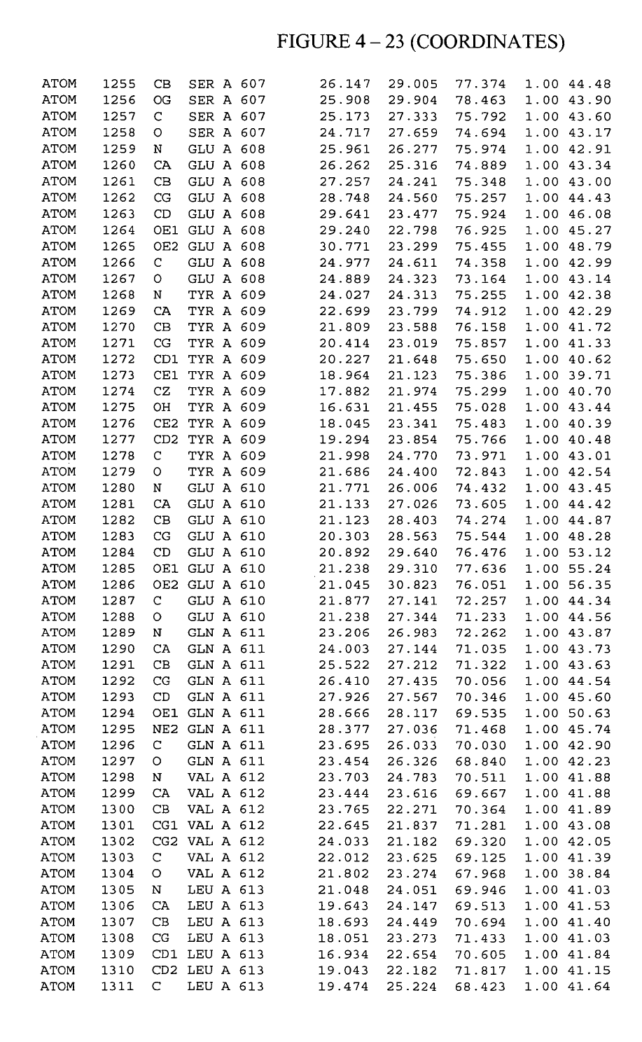

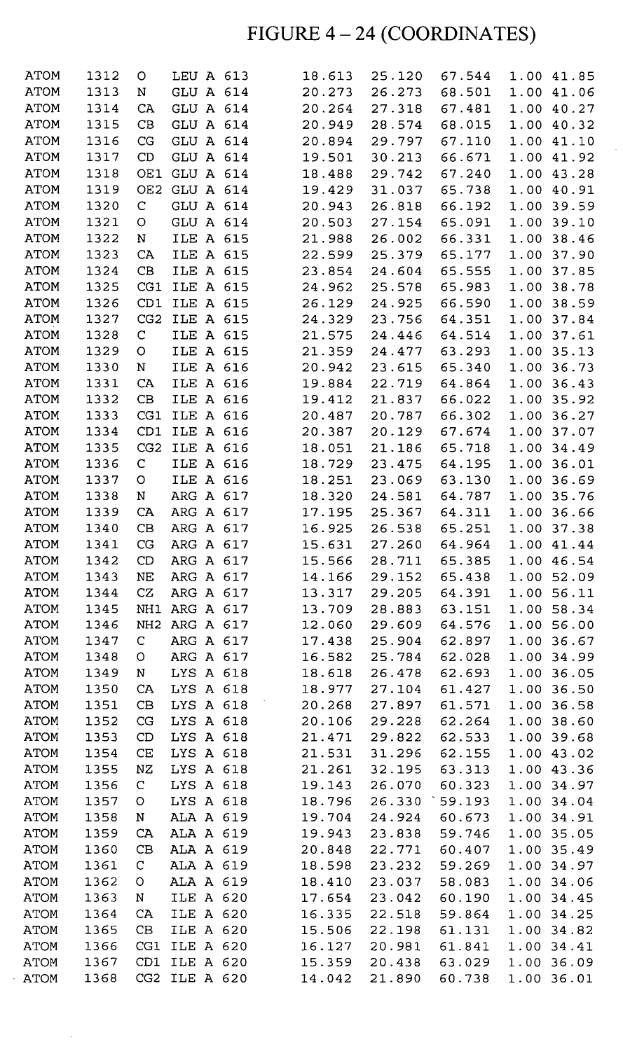

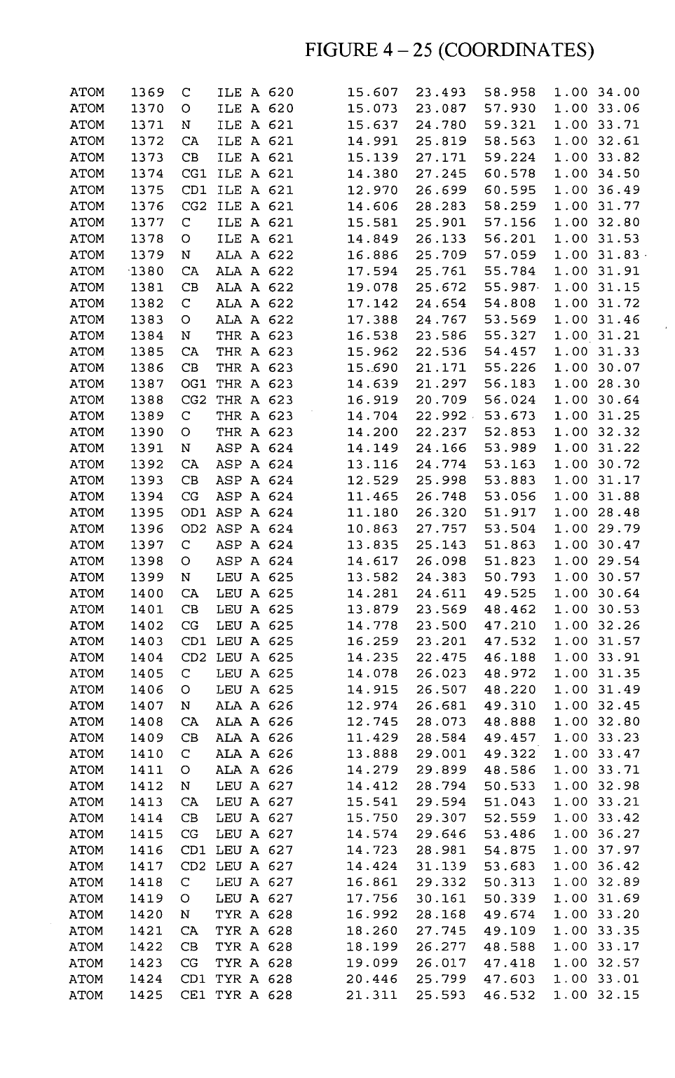

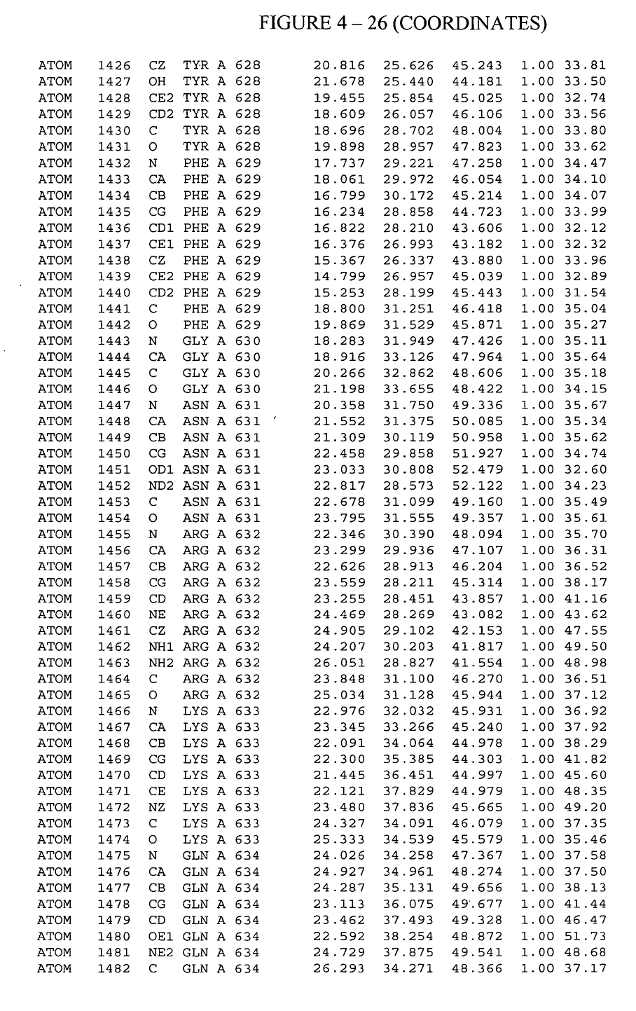

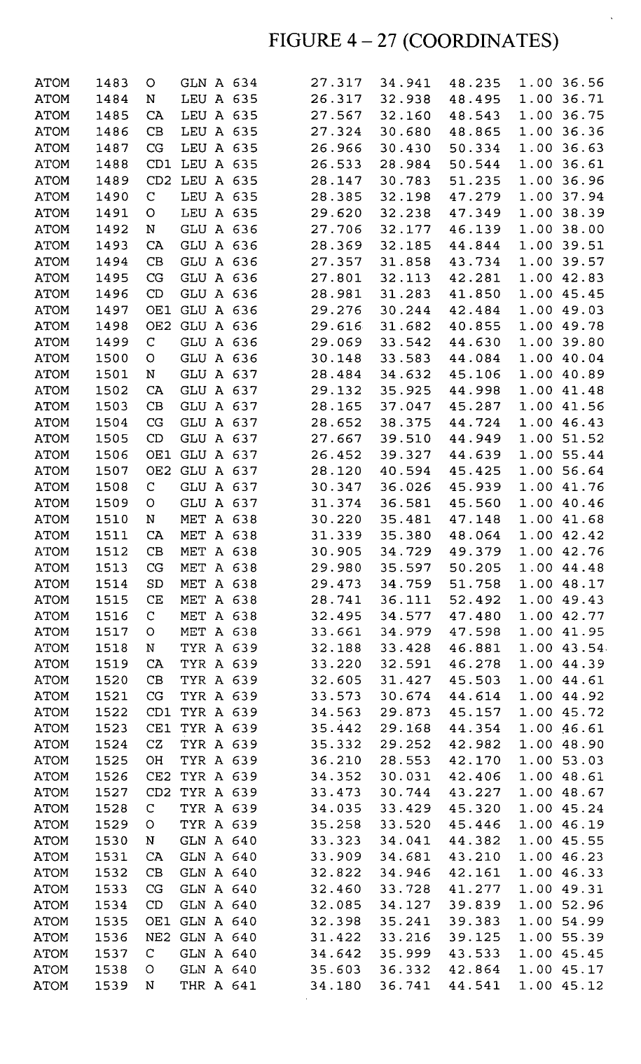

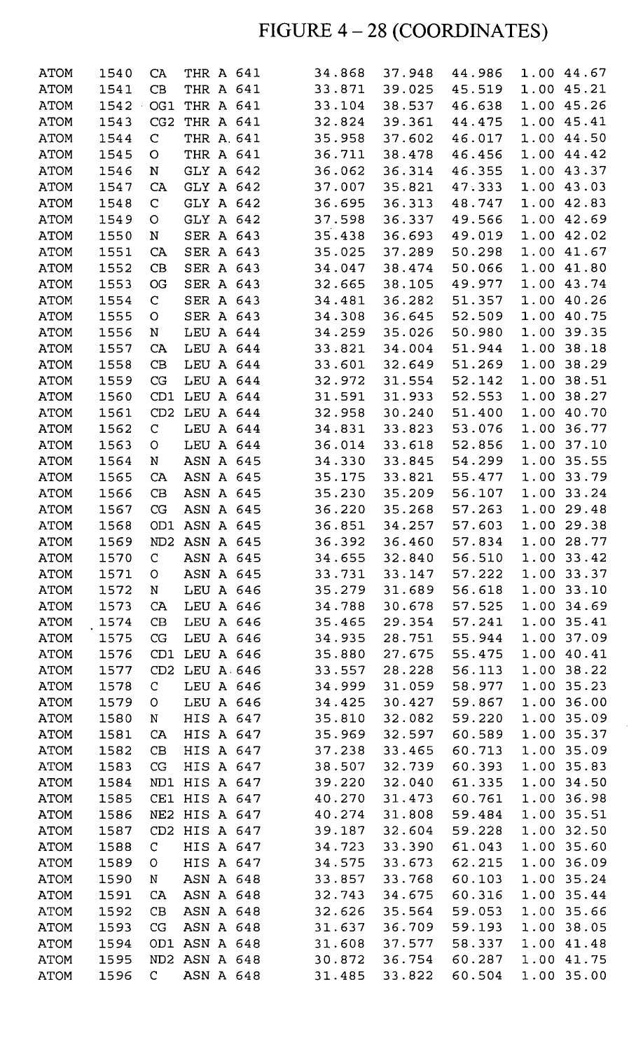

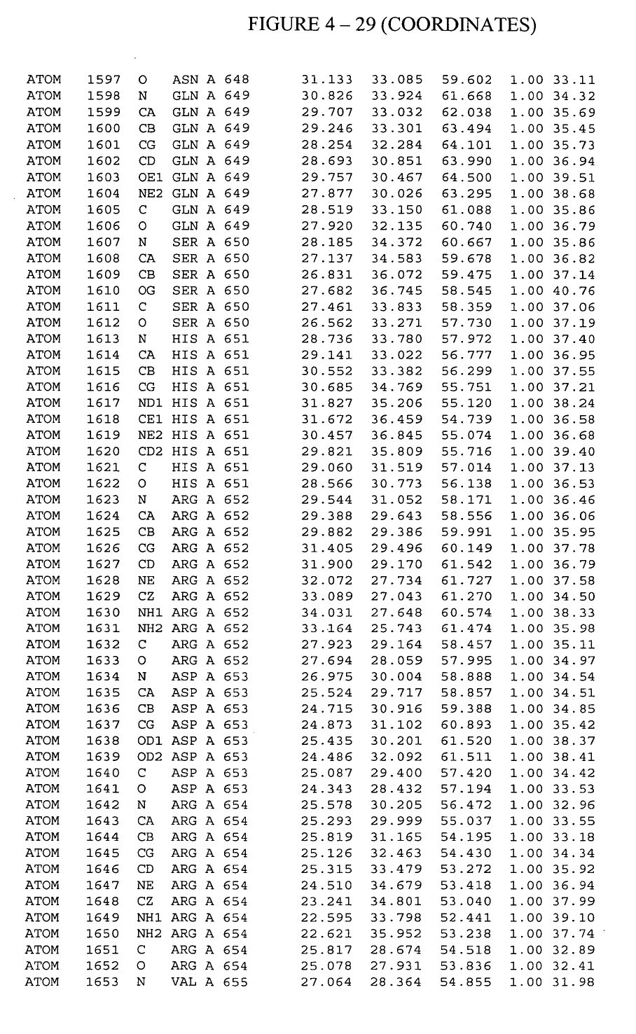

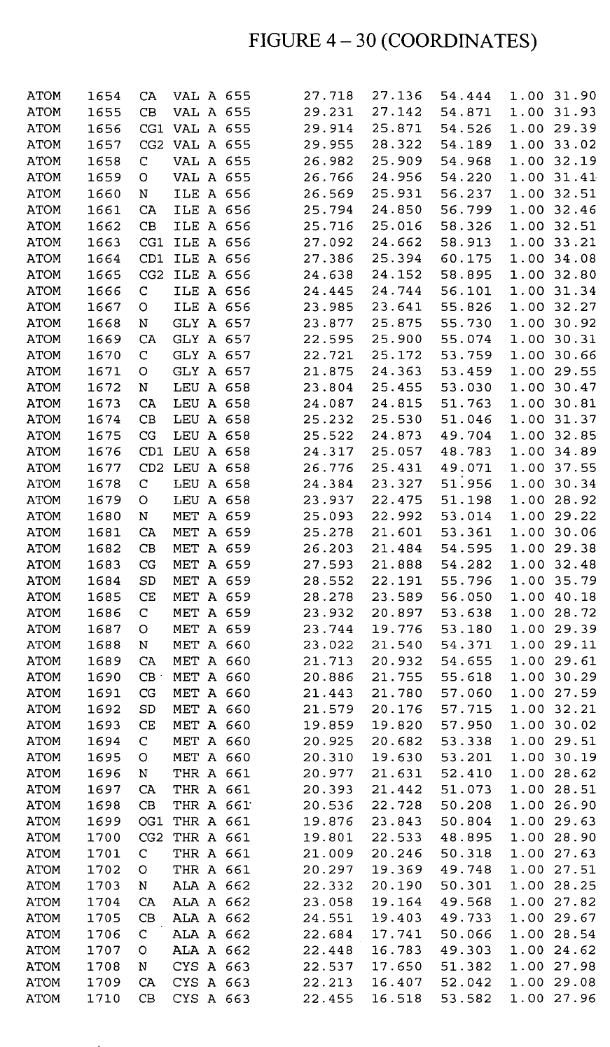

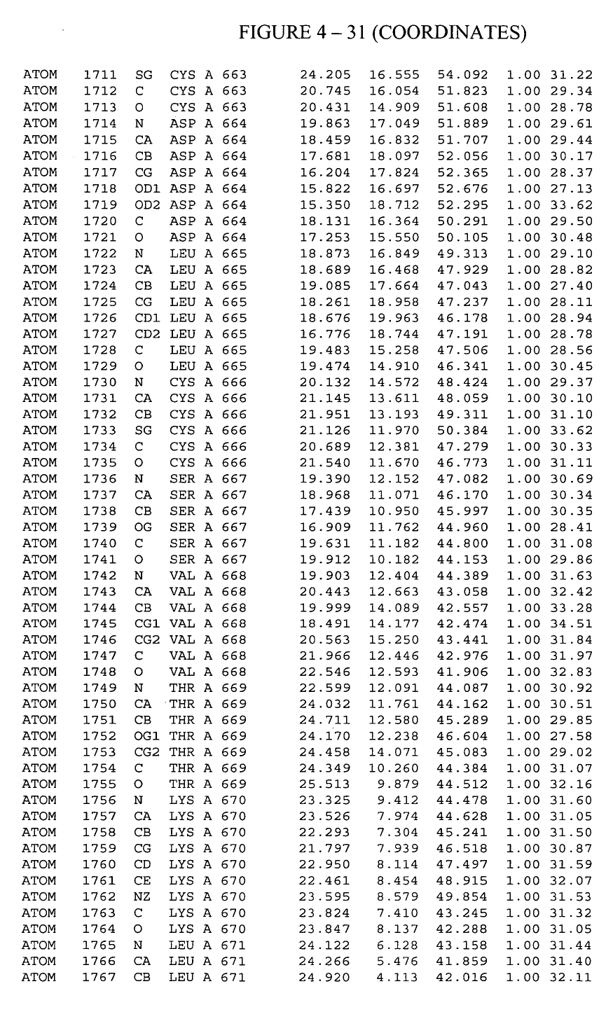

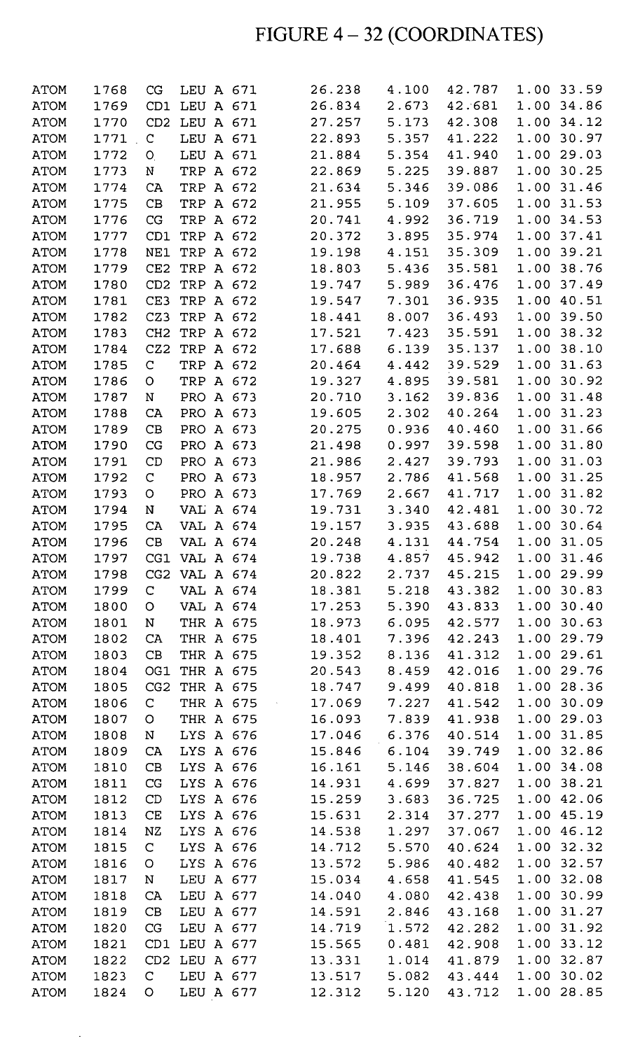

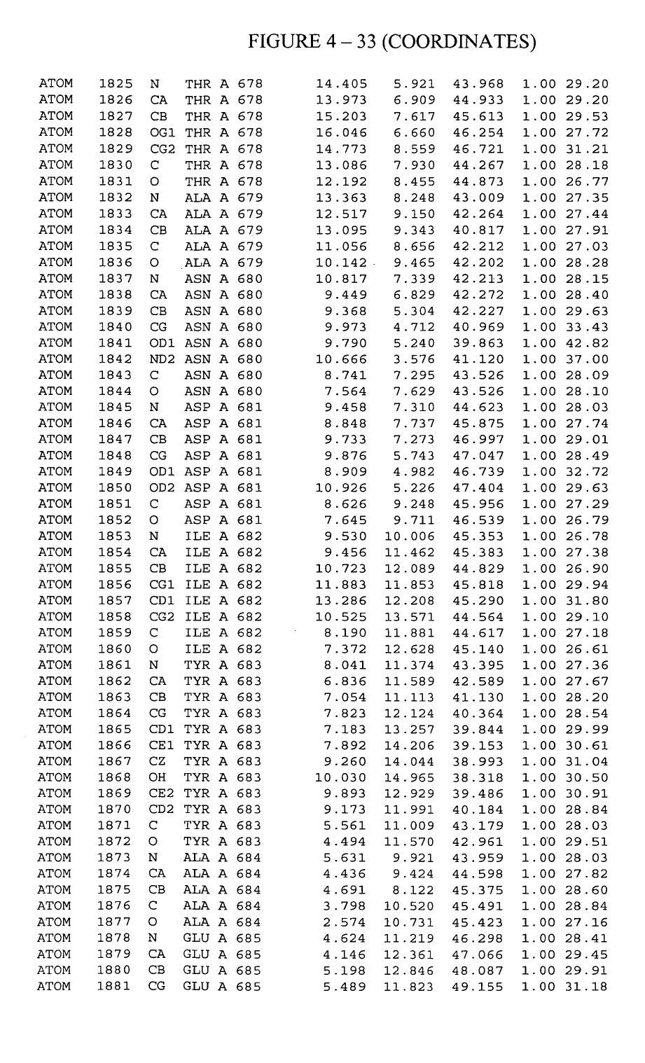

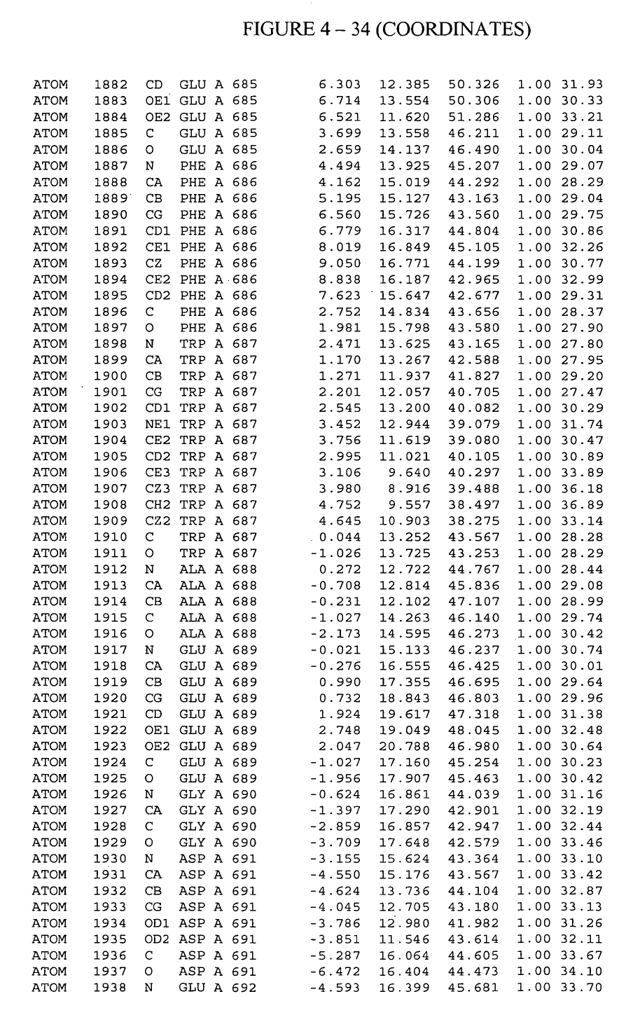

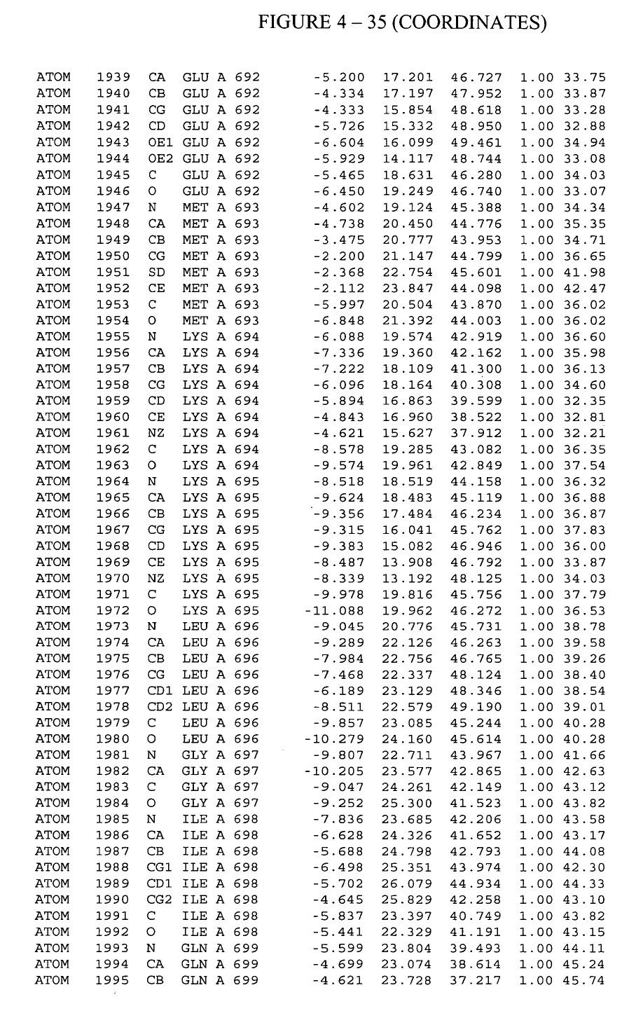

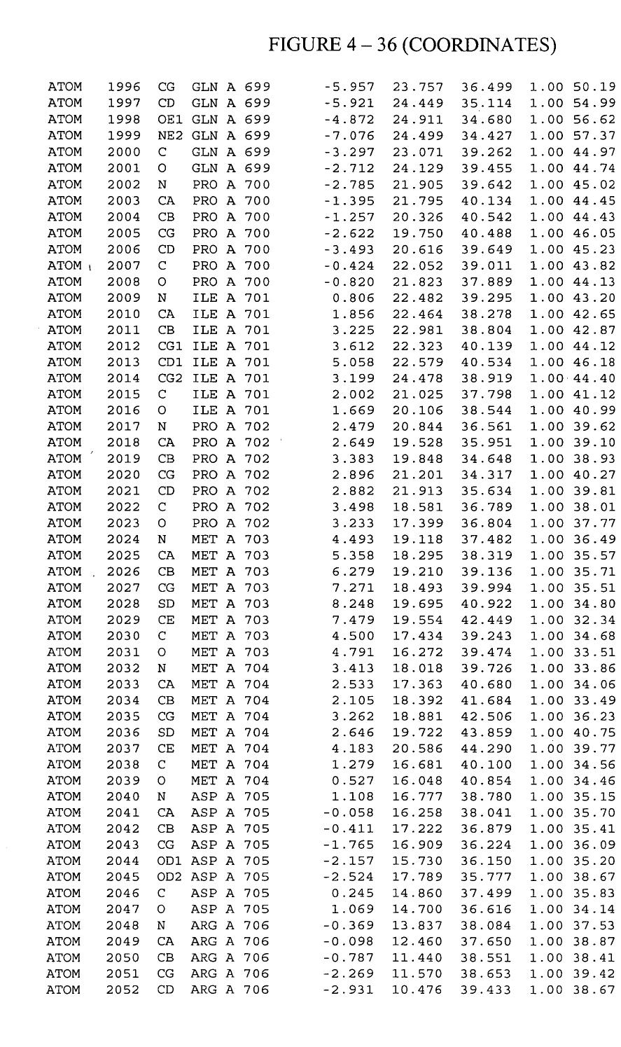

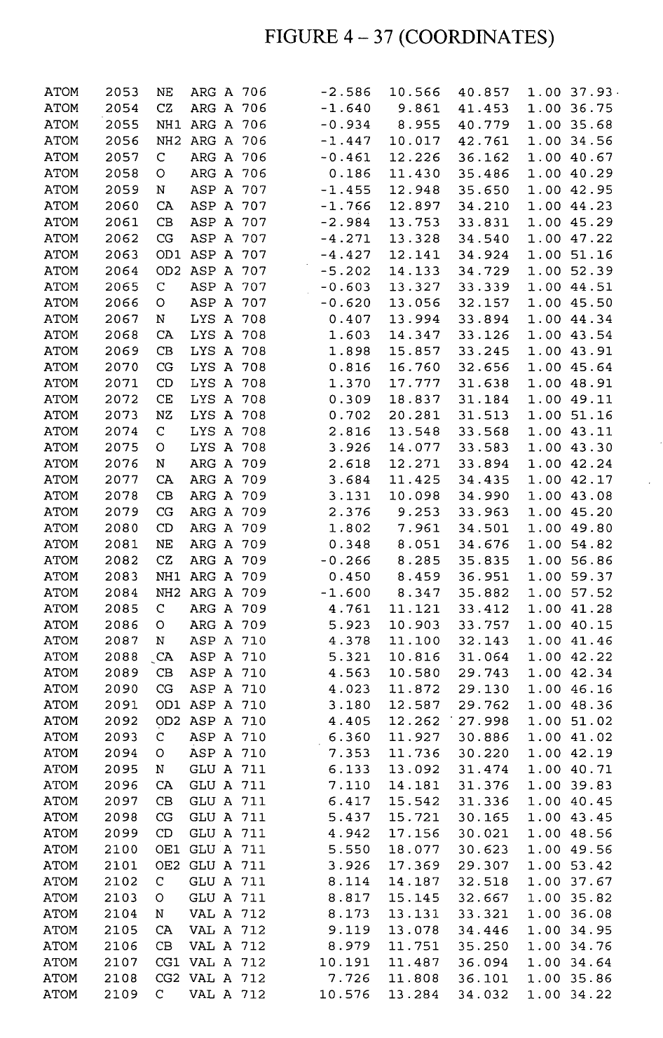

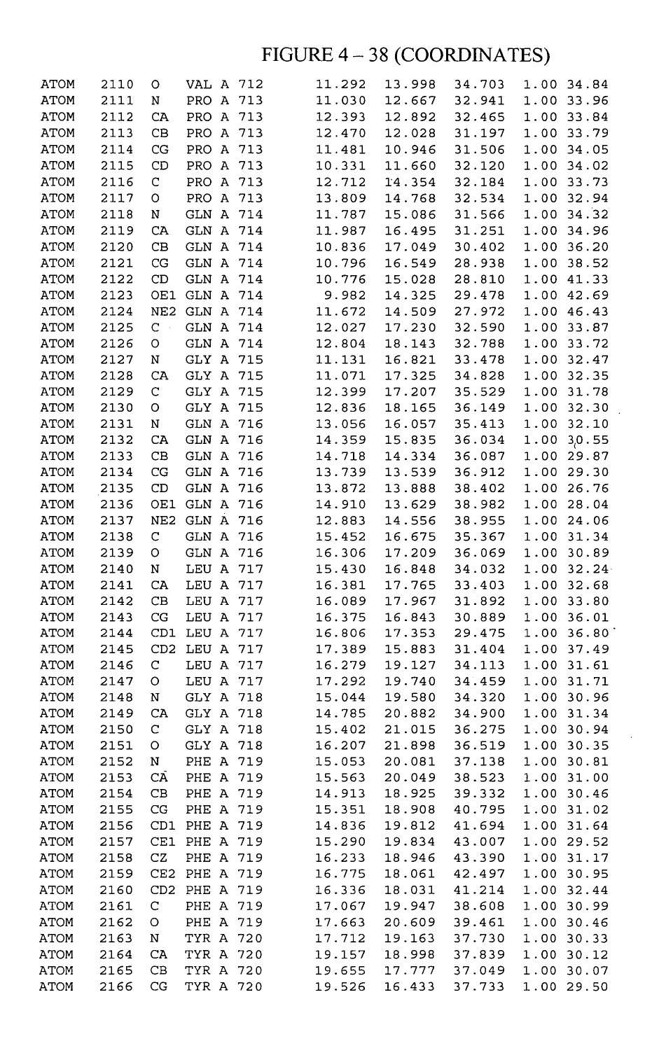

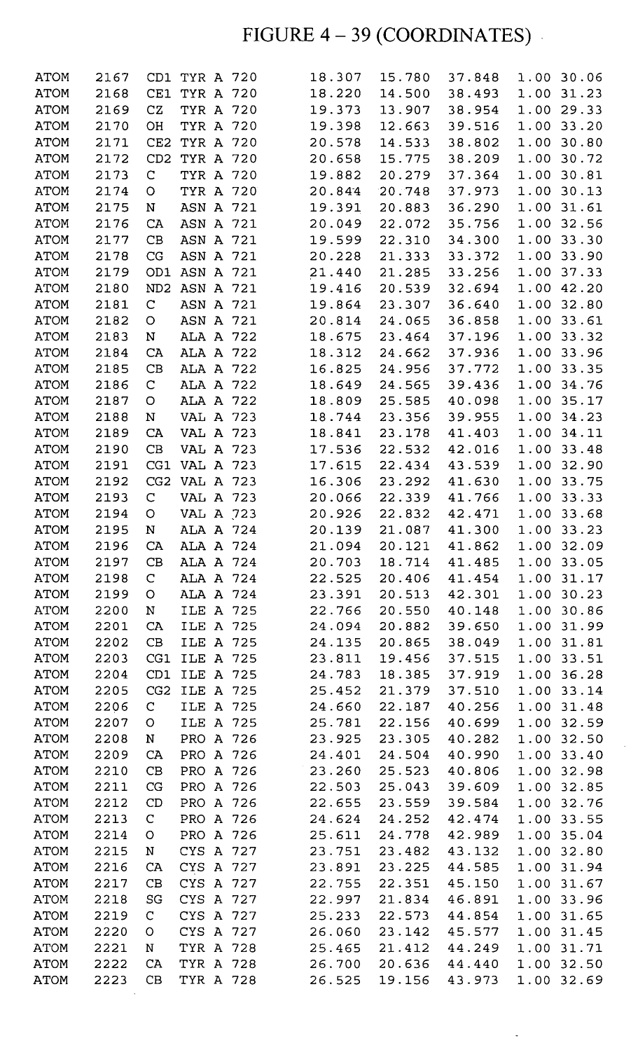

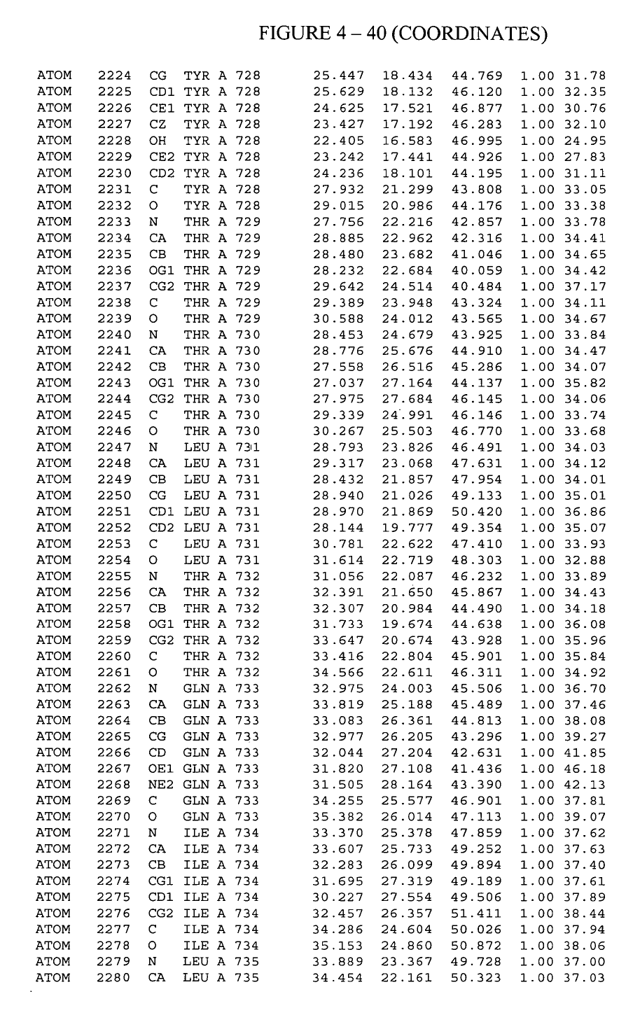

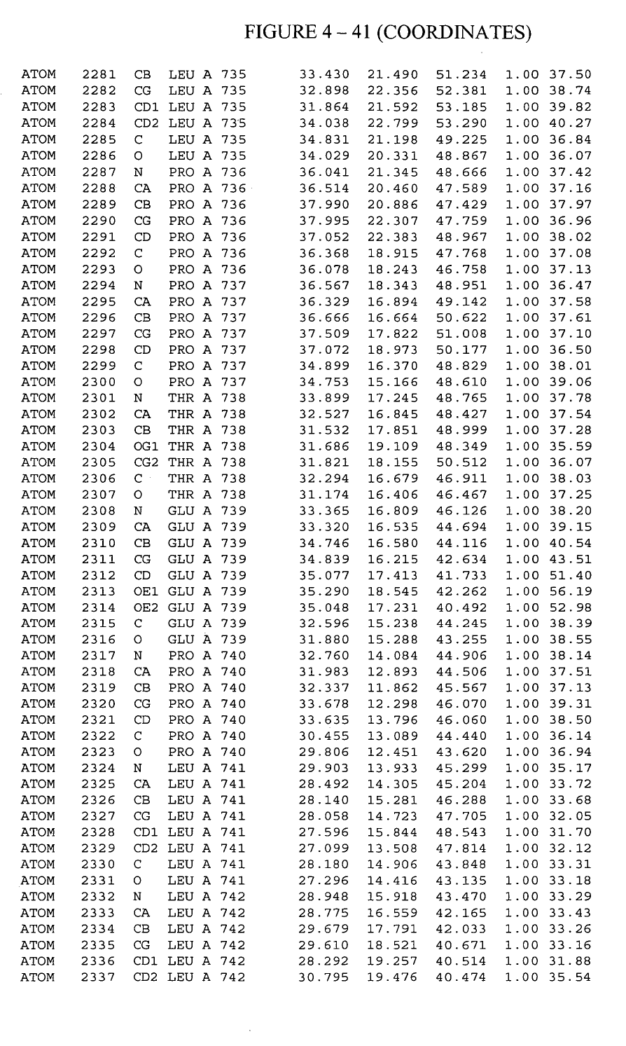

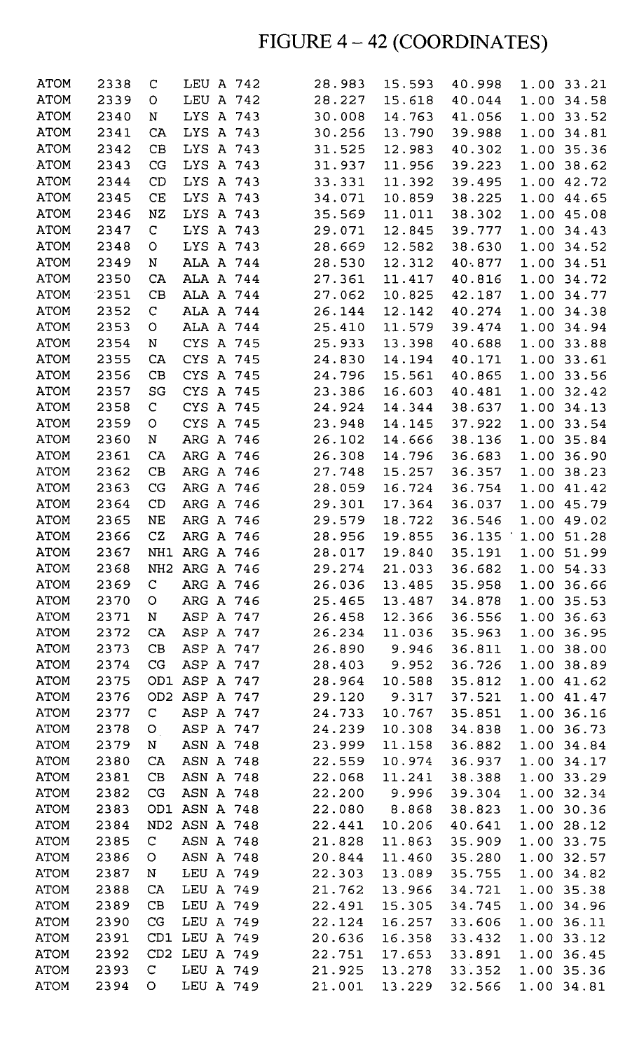

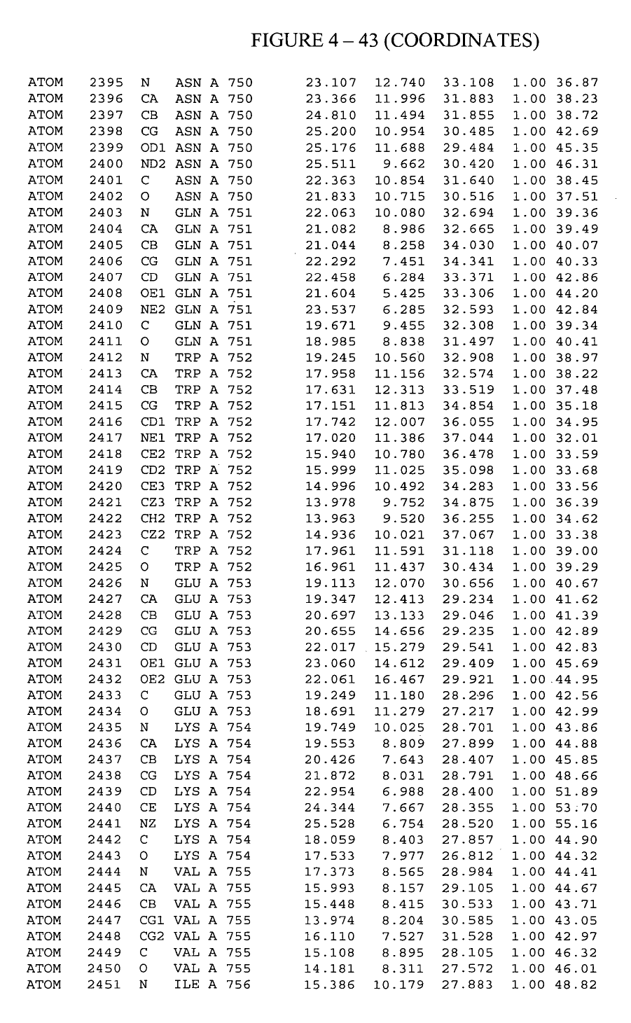

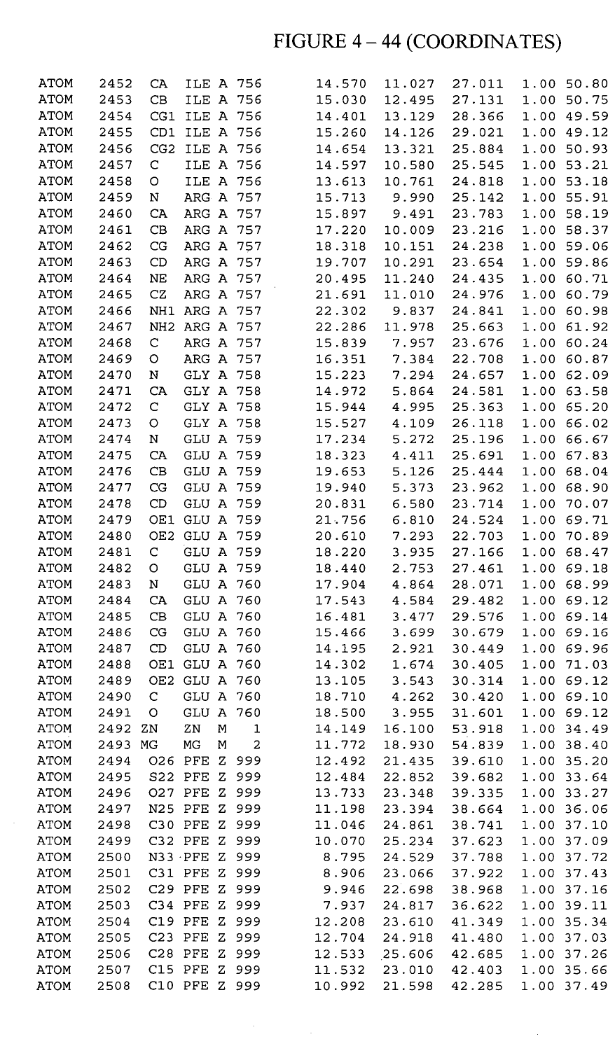

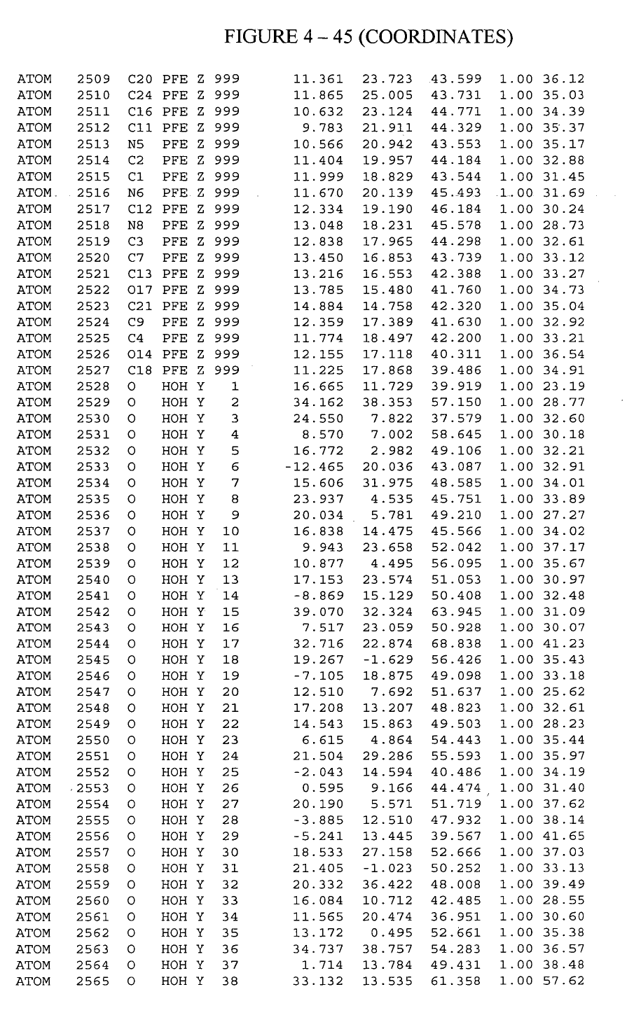

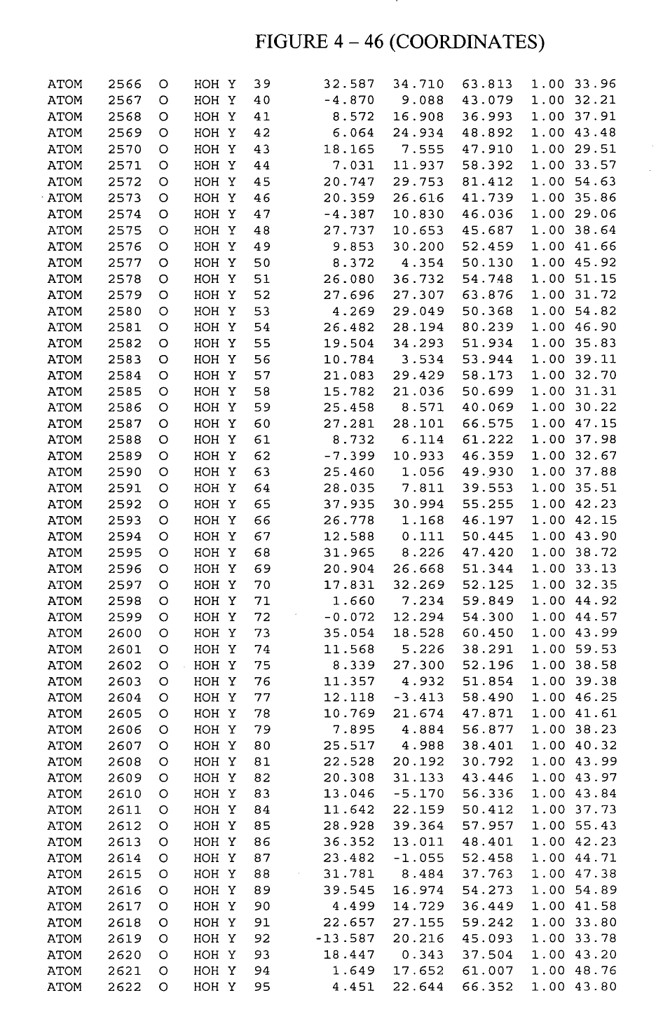

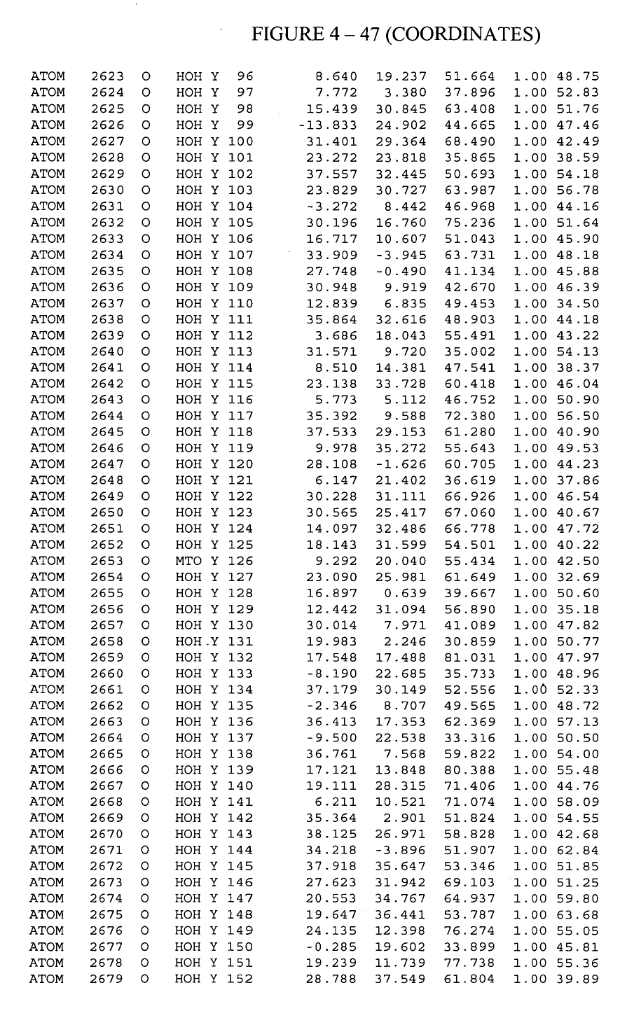

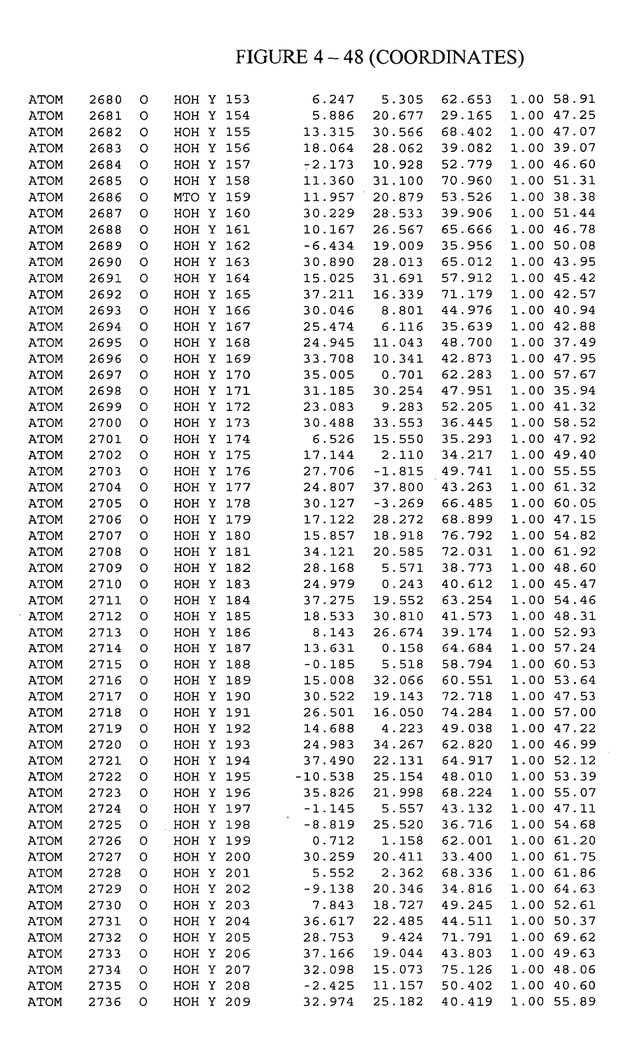

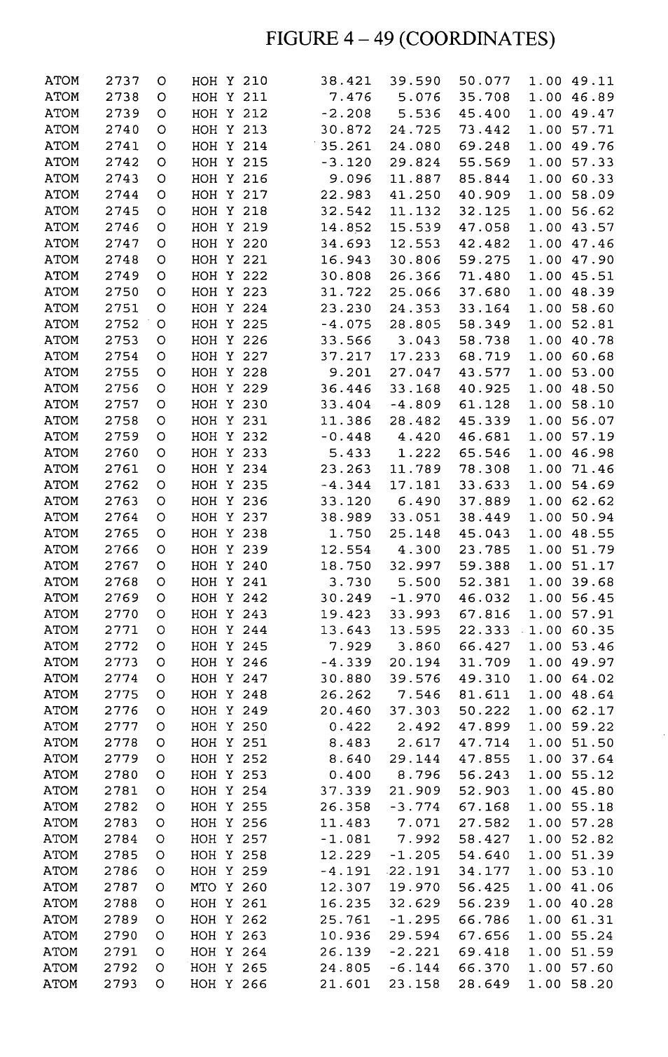

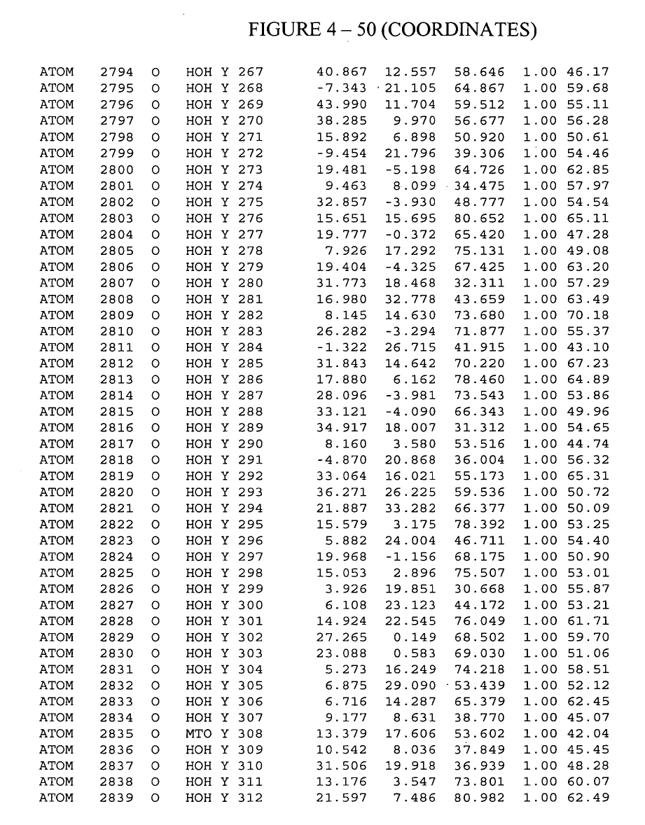

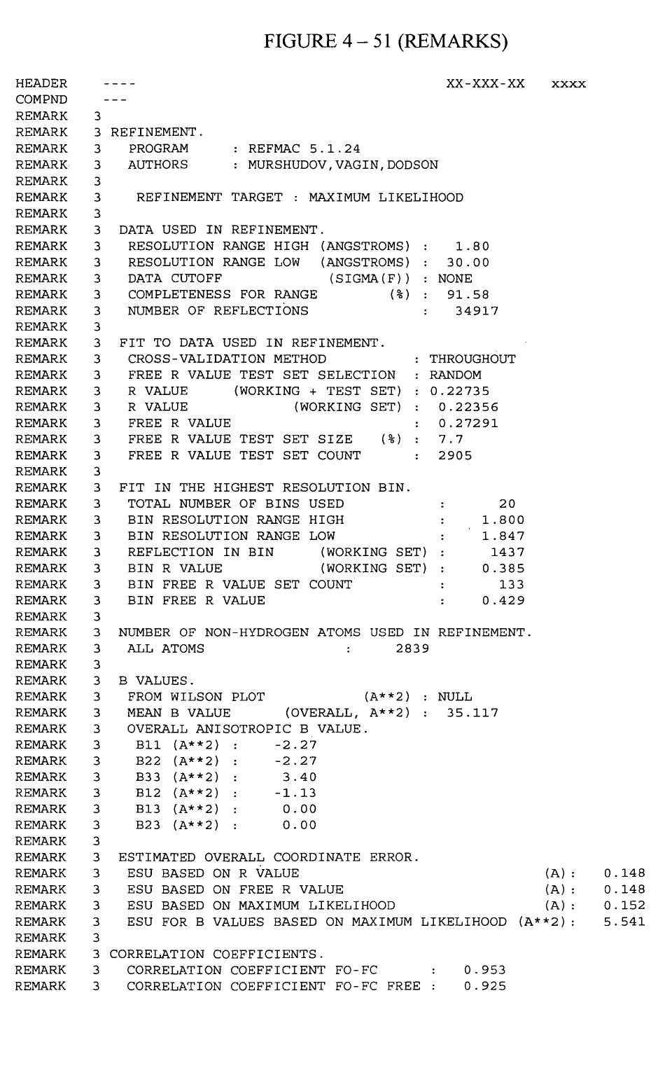

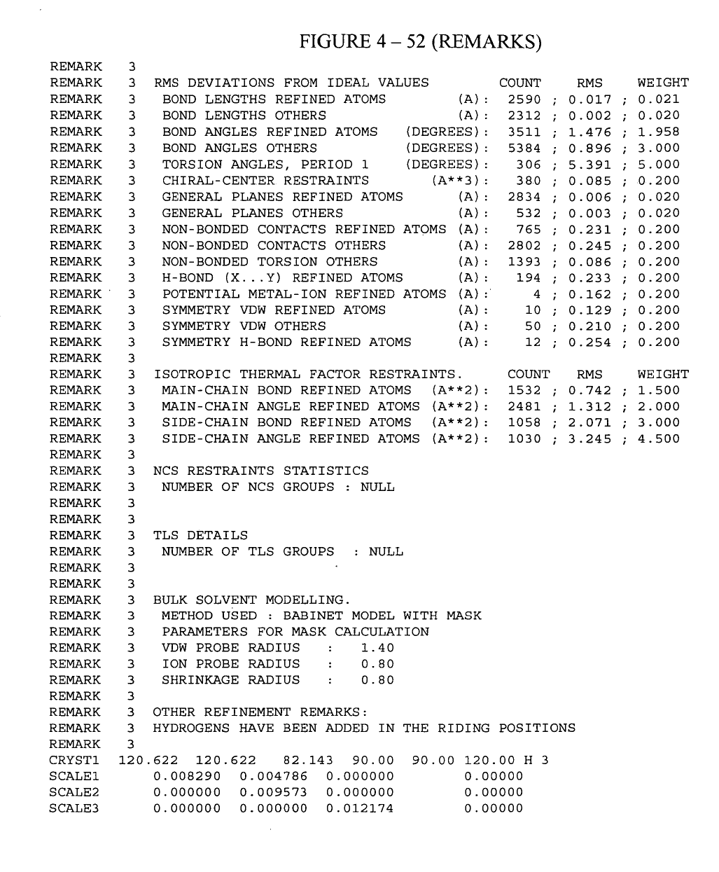

The term “homologue” as used herein means a protein, polypeptide, oliogpeptide, or portion thereof, having preferably at least 90% amino acid sequence identity with PDE10A enzyme as described in SEQ ID No: 1 or SEQ ID No:2 or any catalytic domain described herein, or any functional or structural domain of lipid binding protein. SEQ ID No:1 is a partial amino acid sequence of the wild-type rattus norvegicus (rat) PDE10A. SEQ ID No:2 is the amino acid sequence of the wild type carboxy-terminal catalytic domain of rattus norvegicus (rat) PDE10A that was crystallized in the Examples. While SEQ ID No:3 is the wild-type mus musculus (mouse) PDE10A amino acid sequence, which is at least 90% identical with the PDE10A enzyme as described in SEQ ID No:1. Those of skill in the art understand that a set of structure coordinates determined by X-ray crystallography is not without standard error. As used herein, and for the purpose of this invention, the term “substantially similar atomic coordinates” or atomic coordinates that are “substantially similar” refers to any set of structure coordinates of PDE10A or PDE10A homologues, or PDE10A variants, polypeptide fragments, described by atomic coordinates that have a root mean square deviation for the atomic coordinates of protein backbone atoms (N, Cα, C, and O) of less than about 1.5, 1.2, 1.0, 0.7, 0.5, or even 0.2 Å when superimposed—using backbone atoms—of structure coordinates listed in FIG. 4. For the purpose of this invention, structures that have substantially similar coordinates as those listed in FIG. 4 shall be considered identical to the coordinates listed in FIG. 4. The term “substantially similar” also applies to an assembly of amino acid residues that may or may not form a contiguous polypeptide chain, but whose three dimensional arrangement of atomic coordinates have a root mean square deviation for the atomic coordinates of protein backbone atoms (N, Cα, C, and O), or the side chain atoms, of less than about 1.5, 1.2, 1.0, 0.7, 0.5, or even 0.2 Å when superimposed-using backbone atoms, or the side chain atoms—of the atomic coordinates of similar or the same amino acids from the coordinates listed in FIG. 4. To clarify further, but not intending to be limiting, an example of an assembly of amino acids may be the amino acid residues that form a binding site in an enzyme. These residues may have one or more intervening residues which are distant from the binding site, and therefore may minimally interact with a ligand in the binding sites. In such occurrences, the binding site may be defined for the purpose of structure based drug design as comprising only a handful of amino acid residues. For example in the case of PDE10A, amino acid residues Leu625, Phe629, Val668, Phe686, Met703, Gln716 and Phe719 of SEQ ID NO:1 are known to be near or at the binding site. Thus any molecular assembly that has a root mean square deviation from the atomic coordinates of the protein backbone atoms (N, Cα, C, and O), or the side chain atoms, of one or more of Leu625, Phe629, Val668, Phe686, Met703, Gln716 or Phe719 of SEQ ID NO:1, or any conservative substitutions thereof, of less than about 1.5, 1.2, 1.0, 0.7, 0.5, or even 0.2 Å when superimposed will be considered substantially similar to the coordinates listed in FIG. 4. Those skilled in the art understand that “substantially similar” atomic coordinates are considered identical to the coordinates, or portions thereof, listed in FIG. 4.

Those skilled in the art further understand that the coordinates listed in FIG. 4 or portions thereof may be transformed into a different set of coordinates using various mathematical algorithms without departing from the present invention. For example, the coordinates listed in FIG. 4, or portions thereof, may be transformed by algorithms which translate or rotate the atomic coordinates. Alternatively, molecular mechanics, molecular dynamics or ab intio algorithms may modify the atomic coordinates. Atomic coordinates generated from the coordinates listed in FIG. 4, or portions thereof, using any of the aforementioned algorithms shall be considered identical to the coordinates listed in FIG. 4.

The term “in silico” as used herein refers to experiments carried out using computer simulations. In certain embodiments, the in silico methods are molecular modeling methods wherein 3-dimensional models of macromolecules or ligands are generated. In other embodiments, the in silico methods comprise computationally assessing ligand binding interactions.

The term “ligand” describes any molecule, e.g., protein, peptide, peptidomimetics, oligopeptide, small organic molecule, polysaccharide, polynucleotide, etc., which is designed or developed with reference to the crystal structure of PDE10A as represented by the atomic coordinates listed in FIG. 4. In one aspect the ligand is an agonist, whereby the molecule upregulates (i.e., activate or stimulate, e.g., by agonizing or potentiating) activity, while in another aspect of the invention the ligand is an inhibitor or antagonist, whereby the molecule down-regulates (i.e., inhibit or suppress, e.g. by antagonizing, decreasing or inhibiting) the activity.

The term “modulate” as used herein refers to both upregulation (i.e., activation or stimulation, e.g., by agonizing or potentiating) and down-regulation (i.e., inhibition or suppression, e.g., by antagonizing, decreasing or inhibiting) of an activity.

The term “pharmacophore” as used herein refers to the ensemble of steric and electronic features of a particular structure that is necessary to ensure the optimal supramolecular interactions with a specific biological target structure and to trigger (or to block) its biological response. A pharmacophore may or may not represent a real molecule or a real association of functional groups. In certain embodiments, a pharmacophore is an abstract concept that accounts for the common molecular interaction capacities of a group of compounds towards their target structure. In certain embodiments, the term can be considered as the largest common denominator shared by a set of active molecules. Pharmacophoric descriptors are used to define a pharmacophore, including H-bonding, hydrophobic and electrostatic interaction sites, defined by atoms, ring centers and virtual points. Accordingly, in the context of enzyme ligands, such as for example agonists or antagonists, a pharmacophore may represent an ensemble of steric and electronic factors which are necessary to insure supramolecular interactions with a specific biological target structure. As such, a pharmacophore may represent a template of chemical properties for an active site of a protein/enzyme—representing these properties' spatial relationship to one another—that theoretically defines a ligand that would bind to that site.

The term “precipitant” as used herein is includes any substance that, when added to a solution, causes a precipitate to form or crystals to grow. Examples of precipitants within the scope of this invention include, but are not limited to, alkali (e.g., Li, Na, or K), or alkaline earth metal (e.g., Mg, or Ca) salts, and transition (e.g., Mn, or Zn) metal salts. Common counterions to the metal ions include, but are not limited to, halides, phosphates, citrates and sulfates.

The term “prodrug” as used herein refers to drugs that, once administered, are chemically modified by metabolic processes in order to become pharmaceutically active. In certain embodiments the term also refers to any compound that undergoes biotransformation before exhibiting its pharmacological effects. Prodrugs can thus be viewed as drugs containing specialized non-toxic protective groups used in a transient manner to alter or to eliminate properties, usually undesireable, in the parent molecule.

The term “receptor” as used here in refers to a protein or a protein complex in or on a cell that specifically recognizes and binds to a compound acting as a molecular messenger (neurotransmifter, hormone, lymphokine, lectin, drug, etc.). In a broader sense, the term receptor is used interchangeably with any specific (as opposed to non-specific, such as binding to plasma proteins) drug binding site, also including nucleic acids such as DNA.

The term “recombinant protein” refers to a polypeptide which is produced by recombinant DNA techniques, wherein generally, DNA encoding a polypeptide is inserted into a suitable expression vector which is in turn used to transform a host cell to produce the polypeptide encoded by said DNA. This polypeptide may be one that is naturally expressed by the host cell, or it may be heterologous to the host cell, or the host cell may have been engineered to have lost the capability to express the polypeptide which is otherwise expressed in wild type forms of the host cell. The polypeptide may also be, for example, a fusion polypeptide. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations, including substitutions, deletions and truncation, of a naturally occurring form of the polypeptide.

As used herein, the term “selective PDE10A inhibitor” refers to a substance, for example an organic molecule that effectively inhibits an enzyme from the PDE10A family to a greater extent than any other PDE enzyme, particularly any enzyme from the PDE 1-9 families or any PDE11 enzyme. In one embodiment, a selective PDE10A inhibitor is a substance, for example, a small organic molecule having a Kᵢ for inhibition of PDE10A that is less than about one-half, one-fifth, or one-tenth the Kᵢ that the substance has for inhibition of any other PDE enzyme. In other words, the substance inhibits PDE10A activity to the same degree at a concentration of about one-half, one-fifth, one-tenth or less than the concentration required for any other PDE enzyme. In general a substance is considered to effectively inhibit PDE10A if it has an IC₅₀ or Ki of less than or about 10 mM, 1 mM, 500 nM, 100 nM, 50 nM or even 10 nM.

As used herein the term “small molecules” refers to preferred drugs as they are orally available (unlike proteins which must be administered by injection or topically). Size of small molecules is generally under 1000 Daltons, but many estimates seem to range between 300 to 700 Daltons.

By “therapeutically effective” amount is meant that amount which is capable of at least partially reversing the symptoms of the disease. A therapeutically effective amount can be determined on an individual basis and will be based, at least in part, on a consideration of the species of the mammal, the size of the mammal, the type of delivery system used, and the type of administration relative to the progression of the disease. A therapeutically effective amount can be determined by one of ordinary skill in the art employing such factors and using no more than routine experimentation.

As used herein, the term “transfection” means the introduction of a nucleic acid, e.g., via an expression vector, into a recipient cell by nucleic acid-mediated gene transfer. “Transformation” refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the polypeptide is disrupted.

The term “variants” in relation to the polypeptide sequence in SEQ ID NO:1 or SEQ ID NO:2 include any substitution of, variation of, modification of, replacement of, deletion of, or addition or one or more amino acids from or to the sequence providing a resultant polypeptide sequence for an enzyme having PDE10A activity. Preferably the variant, homologue, fragment or portion, of SEQ ID NO:1 or SEQ ID NO:2, comprise a polypeptide sequence of at least 5 contiguous amino acids, preferably at least 10 contiguous amino acids, preferably at least 15 contiguous amino acids, preferably at least 20 contiguous amino acids, preferably at least 25 contiguous amino acids, or preferably at least 30 contiguous amino acids.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The following amino acid abbreviations are used throughout this disclosure:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanin | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

A. Clones and Expressions

The nucleotide sequence coding for a PDE10A polypeptide, or functional fragment, including the C-terminal peptide fragment of the catalytic domain of PDE10A protein, derivatives or analogs thereof, including a chimeric protein, thereof, can be inserted into an appropriate expression vector, i.e., a vector which contains the necessary elements for the transcription and translation of the inserted protein-coding sequence. The elements mentioned above are termed herein a “promoter.” Thus, the nucleic acid encoding a PDE10A polypeptide of the invention or a functional fragment comprising the C-terminal peptide fragment of the catalytic domain of PDE10A protein, derivatives or analogs thereof, is operationally associated with a promoter in an expression vector of the invention. In preferred embodiments, the expression vector contains the nucleotide sequence coding for the polypeptide comprising the amino acid sequence spanning amino acids Thr442 to Asp774 listed in SEQ ID NO:1. Both cDNA and genomic sequences can be cloned and expressed under the control of such regulatory sequences. An expression vector also preferably includes a replication origin. The necessary transcriptional and translational signals can be provided on a recombinant expression vector. As detailed below, all genetic manipulations described for the PDE10A gene in this section, may also be employed for genes encoding a functional fragment, including the C-terminal peptide fragment of the catalytic domain of the PDE10A protein, derivatives or analogs thereof, including a chimeric protein thereof.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant PDE10A protein of the invention may be expressed chromosomally, after integration of the coding sequence by recombination. In this regard, any of a number of amplification systems may be used to achieve high levels of stable gene expression. (See Sambrook et al., 1989, infra, the pertinent disclosure of which is incorporated by reference herein in its entirety).

A suitable cell for purposes of this invention is one into which the recombinant vector comprising the nucleic acid encoding PDE10A protein is cultured in an appropriate cell culture medium under conditions that provide for expression of PDE10A protein by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques, and in vivo recombination (genetic recombination).

Expression of PDE10A protein may be controlled by any promoter/enhancer element known in the art, but these regulatory elements must be functional in the host selected for expression.

Vectors containing a nucleic acid encoding a PDE10A protein of the invention can be identified by four general approaches: (1) PCR amplification of the desired plasmid DNA or specific mRNA, (2) nucleic acid hybridization, (3) presence or absence of selection marker gene functions, and (4) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., beta.-galactosidase activity, thymidine kinase activity, resistance to antibiotics, transformation phenotype, occlusion body formation in baculovirus, etc.) caused by the insertion of foreign genes in the vector. In another example, if the nucleic acid encoding PDE10A protein is inserted within the “selection marker” gene sequence of the vector, recombinant vectors containing the PDE10A protein insert can be identified by the absence of the PDE10A protein gene function. In the fourth approach, recombinant expression vectors can be identified by assaying for the activity, biochemical, or immunological characteristics of the gene product expressed by the recombinant vector, provided that the expressed protein assumes a functionally active conformation.

A wide variety of host/expression vector combinations may be employed in expressing the DNA sequences of this invention as known by those of skill in the art.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

Vectors can be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

B. Crystal and Space Groups

X-ray structure coordinates define a unique configuration of points in space. Those skilled in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between atomic coordinates remain essentially the same. In addition, a scalable configuration of points can be defined by increasing or decreasing the distances between coordinates by a scalar factor while keeping the angles essentially the same.

One aspect of the present invention relates to a crystalline composition comprising preferably a polypeptide with an amino acid sequence spanning amino acids Thr442 to Asp774 listed in SEQ ID NO:1.

In one embodiment, the present invention discloses a crystalline PDE10A molecule comprising a polypeptide with an amino acid sequence spanning amino acids Thr442 to Asp774 listed in SEQ ID NO:1 complexed with one or more ligands. In another embodiment, the crystallized complex is characterized by the structural coordinates listed in FIG. 4, or portions thereof. In certain embodiments, the atoms of the ligand are within about 4, 7, or 10 angstroms of one or more PDE10A amino acids in SEQ ID NO: 1 preferably selected from Leu625, Phe629, Val668, Phe686, Met703, Gln716 and Phe719. One embodiment of the crystallized complex is characterized as belonging to the R3 space group and has cell dimensions of a=120.6, b=120.6, c=82.1 Å, a=b=90.0, g=120°. This embodiment is encompassed by the structural coordinates of FIG. 4. The ligand may be a small molecule which binds to a PDE10A catalytic domain defined by SEQ ID NO: 2, or portions thereof, with a Ki of less than about 10 mM, 1 mM, 500 nM, 100 nM, 50 nM, or even 10 nM. In a certain embodiment, the ligand is the compound of Formula I, (6,7-Dimethoxy-4-[8-(4-methyl-piperazine-1-sulfonyl)-3,4-dihydro-1H-isoquinolin-2-yl]-quinazoline). In certain embodiments, the ligand is a substrate or substrate analog of PDE10A. In certain embodiments, the ligand(s) may be a competitive or uncompetitive inhibitor of PDE10A. In certain embodiments, the ligand is a covalent inhibitor of PDE10A.

Various computational methods can be used to determine whether a molecule or a binding pocket portion thereof is “structurally equivalent,” defined in terms of its three-dimensional structure, to all or part of PDE10A or its binding pocket(s). Such methods may be carried out in current software applications, such as the molecular similarity application of QUANTA (Accelrys Inc., San Diego, Calif.). The molecular similarity application permits comparisons between different structures, different conformations of the same structure, and different parts of the same structure. The procedure used in molecular similarity to compare structures is divided into four steps: (1) load the structures to be compared; (2) optionally define the atom equivalences in these structures; (3) perform a fitting operation; and (4) analyze the results. Each structure is identified by a name. One structure is identified as the target (i.e., the fixed structure); all remaining structures are working structures (i.e., moving structures). Since atom equivalency within molecular similarity applications is defined by user input, for the purpose of this invention equivalent atoms are defined as protein backbone atoms (N, Cα, C, and O) for all conserved residues between the two structures being compared. A conserved residue is defined as a residue that is structurally or functionally equivalent (See Table 4 infra). In certain embodiments rigid fitting operations are considered. In other embodiments, flexible fitting operations may be considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atoms is an absolute minimum. This number, given in angstroms, is reported by the molecular similarity application.

For the purpose of this invention, any molecule or molecular complex or binding pocket thereof, or any portion thereof, that has a root mean square deviation of conserved residue backbone atoms (N, Cα, C, and O) of less than about 1.5 Å, 1.0 Å, 0.7 Å, 0.5 Å or even 0.2 Å, when superimposed on the relevant backbone atoms described by the reference structure coordinates listed in FIG. 4, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structural coordinates listed in FIG. 4, plus or minus a root mean square deviation from the conserved backbone atoms of those amino acids of not more than 2.0 Å. More preferably, the root mean square deviation is less than about 1.0 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of PDE10A or a binding pocket portion thereof, as defined by the structural coordinates of PDE10A described herein.

The refined x-ray coordinates of the catalytic domain of PDE10A (amino acids 442 to 774 as listed in SEQ ID NO:2), complexed with the compound of Formula 1 (6,7-dimethoxy-4-[8-(4-methyl-piperazine-1-sulfonyl)-3,4-dihydro-1H-isoquinolin-2-yl]-quinazoline), Zn²⁺, Mg²⁺, and 312 water molecules are as listed in FIG. 4.

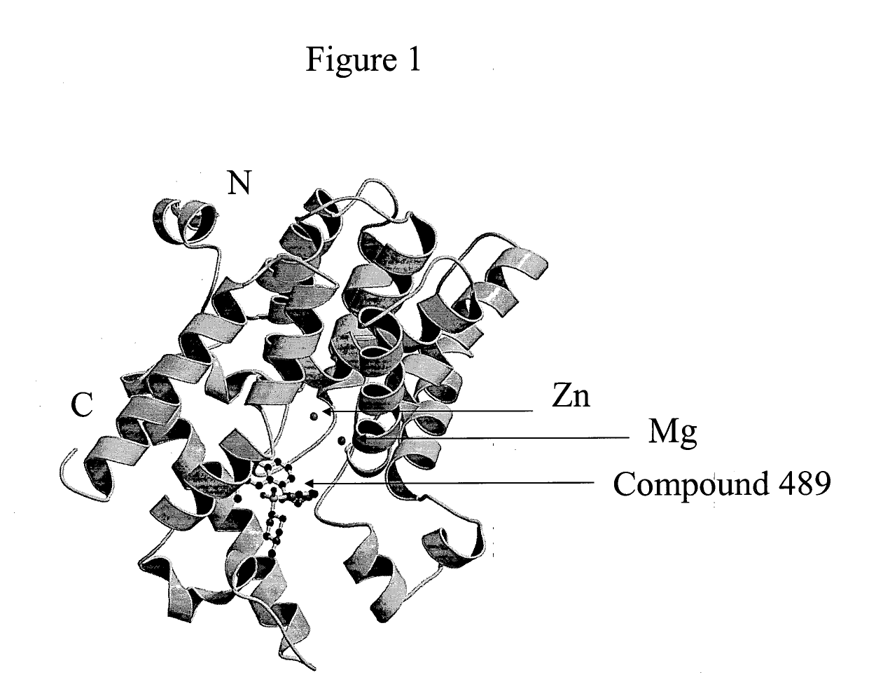

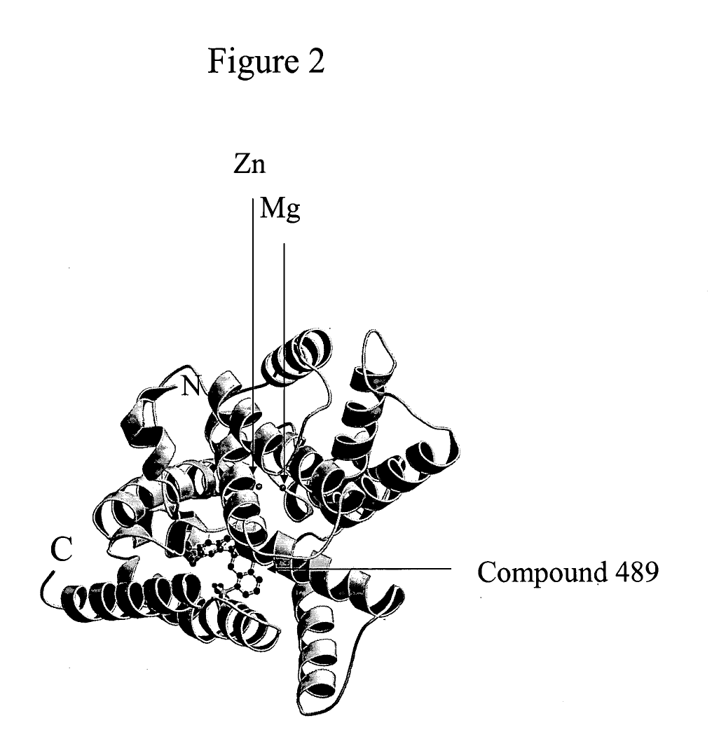

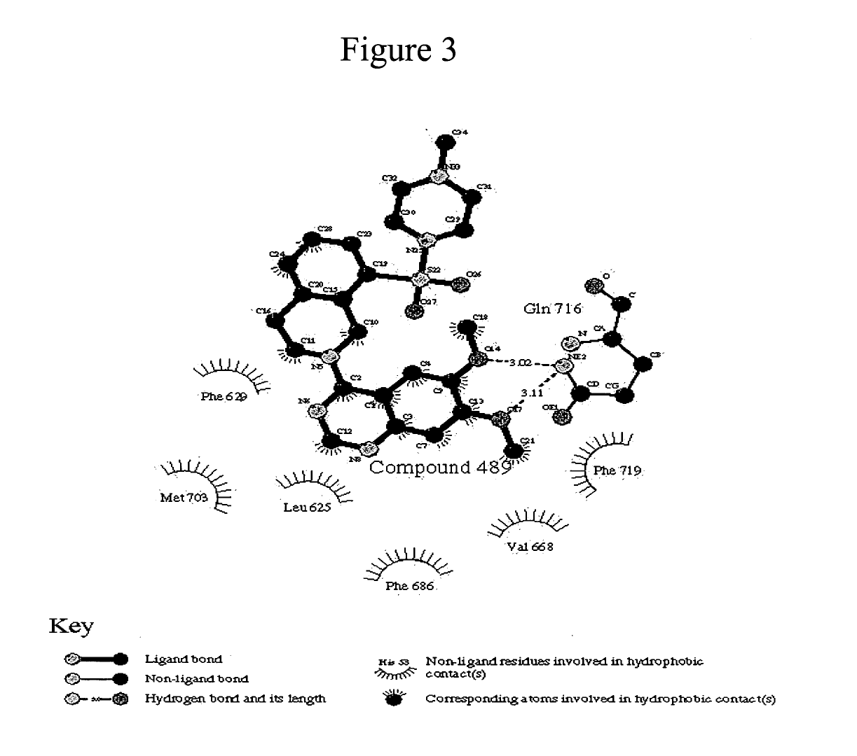

Two orthogonal views of the molecule are shown in FIG. 1 and FIG. 2 and details of the interactions of the inhibitor with protein are shown in FIG. 3.

The structure is composed of a single domain of fourteen a helices and two 3₁₀ helices arranged in a compact fold (FIG. 1). The numbering of the helix is shown below. We have the followed the numbering conversion established by Xu et al., Science, 288:1822-25 (2000), and the start and end points of the helices are determined according to Kabsch and Sander, Biopolymers, 22(12): 2577-637 (1983).

| α helices | Residue range | 3₁₀ helices | Residue range |
|---|---|---|---|
| H1 | 454-461 | A1 | 666-669 |
| H3 | 476-487 | A2 | 702-710 |
| H5 | 495-507 | | |
| H6 | 517-532 | | |
| H7 | 540-552 | | |
| H8 | 562-568 | | |
| H9 | 571-575 | | |
| H10 | 580-594 | | |
| H11 | 606-622 | | |
| H12 | 625-640 | | |
| H13 | 649-664 | | |
| H14 | 672-694 | | |
| H15a | 712-722 | | |
| H15b | 724-734 | | |
| H16 | 739-756 | | |

Two metal ions are in the catalytic site. The first is determined to be Zn²⁺, by analogy with PDE4b, and from an analysis of its coordination geometry. The metal is coordinated by His553 (Nε2-Zn 2.1 Å), His519 (Nε2-Zn 2.0 Å), Asp554 (Oδ2-Zn 2.1 Å), Asp664 (Oδ2-Zn 2.2 Å) and a water molecule (O—Zn 1.7 Å). These residues are completely conserved across the PDE gene family. The second metal ion is coordinated to Asp554 (Oδ1-Zn 1.9 Å) and to a water network that stabilizes the metal environment. Due to the coordination geometry and the relative observed electron density, this second metal ion has been refined as a Mg²⁺ in accordance with a similar observation in the PDE4 structure. (Xu et al., Science, 288:1822-25 (2000)).

One molecule of the inhibitor, 6,7-dimethoxy-4-[8-(4-methyl-piperazine-1-sulfonyl)-3,4-dihydro-1H-isoquinolin-2-yl]-quinazoline. The compound of Formula 1 is seen bound within the active site. The inhibitor binding site is bounded by H14, H15a and H15b on one side, and by the N-terminus of H12 and the two 3₁₀ helices A1 and A2. Protein-inhibitor interactions are shown schematically in the FIG. 2.

The majority of the interactions between the inhibitor and the protein are hydrophobic in nature; with only two hydrogen bonds observed (FIG. 2). Both h-bonds are between Nε2 of the completely conserved Gln716 and the methoxy oxygens O14 (3.0 Å) and O17 (3.1 Å). The quinazoline ring of the inhibitor appears to make a strong π-stacking interaction with Phe719 and an edge-stacking interaction with Phe686. The sulfonamide piperazine group makes no direct interactions with the protein.

Accordingly, the present invention provides a molecule or molecular complex that includes at least a portion of a PDE10A and/or substrate binding pocket. In one embodiment, the PDE10A binding pocket includes the amino acids listed in Table 1, preferably the amino acids listed in Table 2, and more preferably the amino acids listed in Table 3, the binding pocket being defined by a set of points having a root mean square deviation of less than about 1.5, 1.2, 1.0, 0.7, 0.5, or even 0.2 Å, from points representing the backbone atoms of the amino acids in Tables 1-3. In another embodiment, the PDE10A substrate binding pocket includes the amino acids selected from Leu625, Phe629, Val668, Phe686, Met703, Gln716 and Phe719 from SEQ ID NO:1.

TABLE 1
Residues near the binding pocket in PDE10A catalytic domain. Identified
residues are 10 Å away from the compound of Formula 1

| | | | | | | | |
|---|---|---|---|---|---|---|---|
| TYR514 | HIS515 | HIS519 | ASP554 | HIS557 | PHE560 | SER561 | ASN562 |
| SER563 | GLU582 | THR623 | ASP624 | LEU625 | ALA626 | LEU627 | TYR628 |
| PHE629 | GLY630 | THR661 | ASP664 | LEU665 | CYS666 | SER667 | VAL668 |
| THR669 | TRP672 | VAL674 | THR675 | LYS676 | LEU677 | THR678 | ALA679 |
| ASN680 | ASP681 | ILE682 | TYR683 | GLU685 | PHE686 | TRP687 | GLU689 |
| PRO700 | ILE701 | PRO702 | MET703 | MET704 | LYS708 | GLU711 | VAL712 |
| PRO713 | GLN714 | GLY715 | GLN716 | LEU717 | GLY718 | PHE719 | TYR720 |
| ASN721 | ALA722 | VAL723 | ALA724 | ILE725 | PRO726 | TYR728 | CYS745 |
| ASN748 | TRP752 | | | | | | |

TABLE 2
Residues near the binding pocket in PDE10A catalytic domain.
Identified residues are 7 Å away from the compound of Formula 1

| | | | | | | | |
|---|---|---|---|---|---|---|---|
| TYR514 | HIS515 | SER561 | LEU625 | ALA626 | TYR628 | PHE629 | ASP664 |
| LEU665 | SER667 | VAL668 | THR675 | ALA679 | ILE682 | TYR683 | PHE686 |
| ILE701 | PRO702 | MET703 | MET704 | VAL712 | GLN714 | GLY715 | GLN716 |
| LEU717 | GLY718 | PHE719 | TYR720 | ASN721 | ALA722 | VAL723 | TRP752 |

TABLE 3
Residues near the binding pocket in PDE10A catalytic domain. Identified
residues are 4 Å away from the compound of Formula 1

| | | | | | | |
|---|---|---|---|---|---|---|
| LEU625 | PHE629 | VAL668 | TYR683 | PHE686 | MET703 | GLY715 |
| PHE719 | ALA722 | VAL723 | | | | |

C. Isolated Polypeptides and Variants

One embodiment of the invention describes an isolated polypeptide consisting of a portion of PDE10A which functions as the binding site when folded in the proper 3-D orientation. One embodiment is an isolated polypeptide comprising a portion of PDE10A, wherein the portion starts at about amino acid residue Thr442, and ends at about amino acid residue Asp774 as described in SEQ ID NO:1, or a sequence that is at least 90%, 95%, or 98% homologous to a polypeptide with an amino acid sequence spanning amino acids Thr442 to Asp774 as listed in SEQ ID NO:1, such as, for example the polypsptide of the wild-type mus musculus (mouse) PDE10A enzyme, disclosed in SEQ ID No:3.

Another embodiment of the invention comprises crystalline compositions comprising variants of PDE10A. Variants of the present invention may have an amino acid sequence that is different by one or more amino acid substitutions to the sequence disclosed in SEQ ID NO:1 or SED ID NO:2. Embodiments which comprise amino acid deletions and/or additions are also contemplated. The variant may have conservative changes (amino acid similarity), wherein a substituted amino acid has similar structural or chemical properties, for example, the replacement of leucine with isoleucine. Guidance in determining which and how many amino acid residues may be substituted, inserted, or deleted without adversely affecting biological or proposed pharmacological activity may be reasonably inferred in view of this disclosure, and may further be found using computer programs well known in the art, for example, DNAStar® software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Negatively charged amino acids include aspartic acid and glutamic acid; positively charged amino acids include lysine and arginine; amino acids, with uncharged polar head groups having similar hydrophilicity values include leucine, isoleucine, and valine; amino acids with aliphatic head groups include glycine, alanine; asparagine, glutamine, serine; and amino acids with aromatic side chains include threonine, phenylalanine, and tyrosine.

Examples of conservative substitutions are set forth in Table 4 as follows:

TABLE 4

| Original | Examples of |
|---|---|
| Residue | conservative substitutions |
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

“Homology” is a measure of the identity of nucleotide sequences or amino acid sequences. In order to characterize the homology, subject sequences are aligned so that the highest percentage homology (match) is obtained, after introducing gaps, if necessary, to achieve maximum percent homology. N- or C-terminal extensions shall not be construed as affecting homology. “Identity” per se has an art-recognized meaning and can be calculated using published techniques. Computer program methods to determine identity between two sequences, for example, include DNAStar® software (DNAStar Inc. Madison, Wis.); the GCG® program package (Devereux, J., et al. Nucleic Acids Research (1984) 12(1): 387); BLASTP, BLASTN, FASTA (Atschul, S. F. et al., J. Molec Biol (1990) 215: 403). Homology (identity) as defined herein is determined conventionally using the well-known computer program, BESTFIT® (Wisconsin Sequence Analysis Package, Version 8 for Unix, Genetics Computer Group, University Research Park, 575 Science Drive, Madison, Wis., 53711). When using BESTFIT® or any other sequence alignment program (such as the Clustal algorithm from MegAlign software (DNAStar®)) to determine whether a particular sequence is, for example, about 90% homologous to a reference sequence, according to the present invention, the parameters are set such that the percentage of identity is calculated over the full length of the reference nucleotide sequence or amino acid sequence and that gaps in homology of up to about 90% of the total number of nucleotides in the reference sequence are allowed.

Ninety percent of homology is therefore determined, for example, using the BESTFIT® program with parameters set such that the percentage of identity is calculated over the full length of the reference sequence, e.g., SEQ ID NO:1, and wherein up to 10% of the amino acids in the reference sequence may be substituted with another amino acid. Percent homologies are likewise determined, for example, to identify preferred species, within the scope of the claims appended hereto, which reside within the range of about 90% to 100% homology to SEQ ID NO: 1 as well as the binding site thereof. As noted above, N- or C-terminal extensions shall not be construed as affecting homology. Thus, when comparing two sequences, the reference sequence is generally the shorter of the two sequences. This means that, for example, if a sequence of 50 nucleotides in length with precise identity to a 50 nucleotide region within a 100 nucleotide polynucleotide is compared, there is 100% homology as opposed to only 50% homology.

Although the natural polypeptide of SEQ ID NO: 1 and a variant polypeptide may only possess a certain percentage identity, e.g., 90%, they are actually likely to possess a higher degree of similarity, depending on the number of dissimilar codons that are conservative changes. Conservative amino acid substitutions can frequently be made in a protein without altering either the conformation or function of the protein. Similarity between two sequences includes direct matches as well a conserved amino acid substitutes which possess similar structural or chemical properties, e.g., similar charge as described in Table 4.

Percentage similarity (conservative substitutions) between two polypeptides may also be scored by comparing the amino acid sequences of the two polypeptides by using programs well known in the art, including the BESTFIT program, by employing default settings for determining similarity.

A further embodiment of the invention is a crystal comprising the coordinates of FIG. 4, wherein the amino acid sequence is represented by SEQ ID NO:1. A further embodiment of the invention is a crystal comprising the coordinates of FIG. 4, wherein the amino acid sequence is at least 90%, 95%, or 98% homologous to the amino acid sequence represented by SEQ ID NO:1.

Various methods for obtaining atomic coordinates of structurally homologous molecules and molecular complexes using homology modeling are disclosed in U.S. Pat. No. 6,356,845, which is hereby incorporated by reference in its entirety

D. Structure Based Drug Design

Once the three-dimensional structure of a crystal comprising a PDE10A protein, a functional domain thereof, homologue or variant thereof, is determined, a potential ligand (antagonist or agonist) may be examined through the use of computer modeling using a docking program such as GRAM, DOCK, or AUTODOCK (See for example, Morris et al., J. Computational Chemistry, 19:1639-1662 (1998)). This procedure can include in silico fitting of potential ligands to the PDE10A crystal structure to ascertain how well the shape and the chemical structure of the potential ligand will complement or interfere with the catalytic domain of PDE10A. (Bugg et al., Scientific American, December:92-98 (1993); West et al., TIPS, 16:67-74 (1995)). Computer programs can also be employed to estimate the attraction, repulsion, and steric hindrance of the ligand to the binding site. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with the properties of other proteins. This will minimize potential side-effects due to unwanted interactions with other proteins.

One embodiment of the present invention relates to a method of identifying an agent that binds to a binding site on PDE10A catalytic domain wherein the binding site comprises amino acid residues Leu625, Phe629, Val668, Phe686, Met703, Gln716 and Phe719 of SEQ ID NO: 1 comprising contacting PDE10A with a test ligand under conditions suitable for binding of the test ligand to the binding site, and determining whether the test ligand binds in the binding site, wherein if binding occurs, the test ligand is an agent that binds in the binding site. In certain embodiments, the testing may be carried out in silico using a variety of molecular modeling software algorithms including, but not limited to, DOCK, ALADDIN, CHARMM simulations, AFFINITY, C2-LIGAND FIT, Catalyst, LUDI, CAVEAT, and CONCORD. (Brooks, et al. CHARMM: a program for macromolecular energy, minimization, and dynamics calculations. J Comp. Chem 1983, 4:187-217; E. C. Meng, B. K. Shoichet & I. D. Kuntz. Automated docking with grid-based energy evaluation. J Comp Chem 1992, 13:505-524.

In another embodiment, a potential ligand may be obtained by screening a random peptide library produced by a recombinant bacteriophage for example, (Scott and Smith, Science, 249:386-390 (1990); Cwirla et al., Proc. Natl. Acad. Sci., 87:6378-6382 (1990); Devlin et al., Science, 249:404-406 (1990)) or a chemical library, or the like. A ligand selected in this manner can be then be systematically modified by computer modeling programs until one or more promising potential ligands are identified. Such analysis has been shown to be effective in the development of HIV protease inhibitors. (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)).

Such computer modeling allows the selection of a finite number of rational chemical modifications, as opposed to the countless number of essentially random chemical modifications that could be made, of which any one might lead to a useful drug. Each chemical modification requires additional chemical steps, which while being reasonable for the synthesis of a finite number of compounds, quickly becomes overwhelming if all possible modifications needed to be synthesized are actually synthesized. Thus, through the use of the three-dimensional structure disclosed herein and computer modeling, a large number of these compounds can be rapidly screened on a computer monitor screen, and a few likely candidates can be determined without the laborious synthesis of untold numbers of compounds.

Once a potential ligand (agonist or antagonist) is identified, it can be either selected from a library of chemicals as are commercially available from most large chemical companies or alternatively the potential ligand may be synthesized de novo. As mentioned above, the de novo synthesis of one or even a relatively small group of specific compounds is reasonable in the art of drug design. The potential ligand can be placed into any standard binding assay as well known to those skilled in the art to test its effect on PDE10A activity.

When a suitable drug is identified, a supplemental crystal can be grown comprising a protein-ligand complex formed between a PDE10A protein and the drug. Preferably the crystal effectively diffracts X-rays allowing the determination of the atomic coordinates of the protein-ligand complex to a resolution of less than 5.0 Angstroms, more preferably less than 3.0 Angstroms, and even more preferably less than 2.0 Angstroms. The three-dimensional structure of the supplemental crystal can be determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR and AMORE (J. Navaza, Acta Crystallographics ASO, 157-163 (1994)). Once the position and orientation are known, an electron density map can be calculated using the search model to provide X-ray phases. Thereafter, the electron density is inspected for structural differences, and the search model is modified to conform to the new structure. Using this approach, it is possible to use the claimed structure of PDE10A to solve the three-dimensional structures of any such PDE10A complexed with a new ligand. Other computer programs that can be used to solve the structures of such STAT crystals include QUANTA; CHARMM; INSIGHT; SYBYL; MACROMODEL; and ICM.

Various in silico methods for screening, designing or selecting ligands are disclosed in U.S. Pat. No. 6,356,845, the pertinent disclosure of which is incorporated by reference herein.

E. Ligands

In one aspect, the present invention discloses ligands which interact with a binding site of the PDE10A catalytic domain defined by a set of points having a root mean square deviation of less than about 2.0 Å from points representing the backbone atoms of the amino acids represented by the structure coordinates listed in FIG. 4. A further embodiment of the present invention comprises binding agents which interact with a binding site of PDE10A defined by a set of points having a root mean square deviation of less than about 2.0, 1.7, 1.5, 1.2, 1.0, 0.7, 0.5, or even 0.2 Å from points representing the backbone atoms of the amino acids represented by the structure coordinates listed in FIG. 4. Such embodiments represent variants of the PDE10A crystal.

In another aspect, the present invention describes ligands which bind to a correctly folded polypeptide comprising an amino acid sequence spanning amino acids 442 to 774 listed in SEQ ID NO:1, or a homologue-or variant thereof. In certain embodiments, the ligand is a competitive or uncompetitive inhibitor of PDE10A. In certain embodiments the ligand inhibits PDE10A with an IC₅₀ or Ki of less than about 10 mM, 1 mM, 500 nM, 100 nM, 50 nM or 10 nM. In certain embodiments, the ligand inhibits PDE10 with a Kᵢ that is less than about one-half, one-fifth, or one-tenth the Kᵢ that the substance has for inhibition of any other PDE enzyme. In other words, the substance inhibits PDE10A activity to the same degree at a concentration of about one-half, one-fifth, one-tenth or less than the concentration required for any other PDE enzyme.

One embodiment of the present invention relates to ligands, such as proteins, peptides, peptidomimetics, small organic molecules, etc., designed or developed with reference to the crystal structure of PDE10A as represented by the coordinates presented herein in FIG. 4, and portions thereof. Such binding agents interact with the binding site of the PDE10A represented by one or more amino acid residues selected from Leu625, Phe629, Val668, Phe686, Met703, Gln716 and Phe719.

F. Machine Readable Storage Media

Transformation of the structure coordinates for all or a portion of PDE10A, or the PDE10A/ligand complex or one of its binding pockets, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium comprising a data storage material encoded with machine readable data which, when using a machine programmed with instructions for using said data, is capable of displaying a graphical three-dimensional representation of any of the molecule or molecular complexes of this invention that have been described above. In a preferred embodiment, the machine-readable data storage medium comprises a data storage material encoded with machine readable data which, when using a machine programmed with instructions for using said data, is capable of displaying a graphical three-dimensional representation of a molecule or molecular complex comprising all or any parts of a PDE10A C-terminal catalytic domain or binding pocket, as defined above. In another preferred embodiment, the machine-readable data storage medium is capable of displaying a graphical three-dimensional representation of a molecule or molecular complex defined by the structure coordinates of the amino acids listed in FIG. 4, plus or minus a root mean square deviation from the backbone atoms of said amino acids of not more than 2.0 Å.

In an alternative embodiment, the machine-readable data storage medium comprises a data storage material encoded with a first set of machine readable data which comprises the Fourier transform of the structural coordinates set forth in FIG. 4, and which, when using a machine programmed with instructions for using said data, can be combined with a second set of machine readable data comprising the X-ray diffraction pattern of a molecule or molecular complex to determine at least a portion of the structural coordinates corresponding to the second set of machine readable data.

For example, a system for reading a data storage medium may include a computer comprising a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may comprise CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding pocket of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

G. Pharmaceutical Compositions

The present invention contemplates methods for treating certain diseases in a mammal, preferably a human, in need of such treatment using the ligands, and preferably the inhibitors, as described herein. The ligand can be advantageously formulated into pharmaceutical compositions comprising a therapeutically effective amount of the ligand, a pharmaceutically acceptable carrier and other compatible ingredients, such as adjuvants, Freund's complete or incomplete adjuvant, suitable for formulating such pharmaceutical compositions as is known to those skilled in the art. Pharmaceutical compositions containing the ligand can be used for the treatment of a variety of psychotic disorders and condition such as schizophrenia, delusional disorders and drug induced psychosis; anxiety disorders such as panic and obsessive-compulsive disorder; and movement disorders including Parkinson's disease and Huntington's disease.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to selected from Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

The pharmaceutical composition is administered to the mammal in a therapeutically effective amount such that treatment of the disease occurs.

The present invention is further illustrated by the following examples, which should not be construed as limiting in any way. The contents of all cited references (including literature references, issued patents, published patent applications as cited throughout this application) are hereby expressly incorporated by reference in their entireties.

The practice of the present invention will employ, unless otherwise indicated, conventional techniques of cell biology, cell culture, molecular biology, microbiology and recombinant DNA, X-ray crystallography, and molecular modeling which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Crystallography Made Clear: A Guide For Users Of Macromolecular Models (Gales Rhodes, 2^{nd} Ed. San Diego: Academic Press, 2000).

### EXAMPLES

### Example 1

### Construction and Expression of His6-tagged PDE10A Wild Type Catalytic Domain

Amino acids 442-774 of rattus norvegicus (rat) wild type PDE10A (SEQ ID NO:1) were subcloned into a into pFastBac-1 in order to generate recombinant baculovirus using the Bac-to-Bac system (Gibco Carlsbad, Calif.), corresponding to the amino acids in SEQ ID NO:2. The modified fusion protein was expressed in SF21, as a His-tagged version, with amino acids 2-7 of SEQ ID NO:2 being the HIS-tag portion, and amino acids 20-362 of SEQ ID NO:2 (Thr442 to Asp774 of SEQ ID NO:1) being the catalytic region of the rat PDE10A protein portion. The insect cells were infected with the recombinant baculovirus at a MOI (multiplicity of infection) of 0.5 and harvested 72 hrs. post infection. Pellets of infected cells were frozen at −80° C. for transfer to purification.

### Example 2

### Purification of His6-tagged PDE10A Wild Type Catalytic Domain

Baculovirus cell paste (80 g) containing the over expressed PDE10a N3C2 recombinant protein was resuspended in 6 volumes (˜430 ml) buffer A, containing 50 mM HEPES (4-(2-hydroxyethyl)-1-piperazineethanesulfonic acid) pH 7.5, 300 mM NaCl (sodium chloride), 3% (v/v) glycerol, 0.1 mM TCEP (Tri(2-carboxyethyl) phosphine hydrochloride) and Complete™ protease inhibitor cocktail tablets (Roche). The cells were lysed with one pass on a microfluidizer and the cell debris was removed by centrifugation at 4° C. for 45 minutes at 14,000 rpm in a Sorval SLA-1500 rotor. The supernatant was transferred to a clean tube and 10 ml of TALON Metal Affinity Resin (BD-Clonetech) was added. The suspension was incubated with gentle rocking at 4° C. for 1 hour and then subjected to centrifugation at 700×g in a swinging bucket rotor. The supernatant was discarded and the resin was resuspended in 20 ml buffer A and transferred to a XK-16 column (Pharmacia Skokie, Ill.) connected to an FPLC™. The resin was washed with 5 column volumes of buffer A. Following the wash step, the column containing the bound resin was connected upstream to a XK-26/30 Fast Desalt column (Pharmacia) previously equilibrated with buffer A. The PDE10A N3C2 was eluted from the TALON resin with a step gradient of buffer A+120 mM Imidizole. The eluted fractions were buffer exchanged into buffer B containing 25 mM HEPES pH 7.5, 50 mM NaCl, 0.1 mM TCEP, 10 μM E64 (trans-Epoxysuccinyl-L-leucylamido(4-guanidino)butane N-(trans-Epoxysuccinyl)-L-leucine 4-guanidinobutylamide L-trans-3-Carboxyoxiran-2-carbonyl-L-leucylagmatine), 1 mM PMSF (phenylmethylsulfonyl fluoride), 1 μg/ml leupeptin and loaded onto a monoS column (Pharmacia). The protein was eluted with a gradient from 0-500 mM NaCl over 40 column volumes in buffer B. The eluted fraction was concentrated to 1.0 ml and loaded onto a Superdex 75 HiLoad 16/60 prep grade column (Pharmacia) equilibrated with buffer C containing 25 mM HEPES pH 7.5, 150 mM NaCl, 1 mM TCEP, 10 μM E64, 1 mM PMSF, 1 μg/ml leupeptin. The protein eluted between 60-70 ml. The eluted fraction was concentrated to 5.7 mg/ml.

### Example 3

### Crystallization of PDE10A Wild-type Catalytic Domain with Compound of Formula 1 (6,7-dimethoxy-4-[8-(4-methyl-piperazine-1-sulfonyl)-3,4-dihydro-1H-isoquinolin-2-yl]-quinazoline)

Crystallization screens were setup using the sitting drop/vapor diffusion method in 96-well Greiner plates. The protein was mixed 2:1 with the compound of Formula 1 (6,7-dimethoxy-4-[8-(4-methyl-piperazine-1-sulfonyl)-3,4-dihydro-1H-isoquinolin-2-yl]-quinazoline). Conditions were screened using the Hampton Research Crystal Screen HT and Emerald Biosciences Wizard I & II screens. Crystals were obtained under condition B5 of the Crystal Screen HT containing 15% PEG-4000, 0.05M Tris pH 8.5, 0.1M lithium sulfate. Optimization of this condition using the hanging drop/vapor diffusion method in 24-well VDX plates produced crystals measuring 0.23×0.25×0.04 mm with well conditions containing 20% PEG-4000 (polyethylene glycol-4000), 0.1M Tris pH 8.5, 0.2M ammonium sulfate.

### Example 4

### X-ray Data Collection, Structure Determination and Refinement of PDE10A: Compound of Formula 1 (6,7-dimethoxy-4-[8-(4-methyl-piperazine-1-sulfonyl)-3,4-dihydro-1H-isoquinolin-2-yl]-quinazoline) complex

The crystals prepared in Example 3 were transferred to a cryoprotectant solution, made up of the reservoir solution, with 15% ethylene glycol, and then flash-frozen in a stream of cold nitrogen gas at 100K. A full data set was collected from one crystal frozen in this manner on a Rigaku RAXIS IIc detector, mounted on a Rigaku RU-200 generator with Osmic optics. Data were processed using the HKL suite of software (Otwinowski, Z. & Minor, W. Methods Enzymology 276, 307-326 (1997). Data collection statistics are summarized in Table 5a.

The crystals belong to space group R3 with unit cell dimensions a=120.6, b=120.6, c=82.1 Å, a=b=90.0, g=120°. They contain 1 molecule of the polypeptide, and one molecule of the inhibitor per asymmetric unit.

The structure was solved by the method of molecular replacement, using the program AMORE (Navaza, J., Acta Cryst., 157-163 (1994)). A homology model of PDE10, based on the previously determined structure of PDE5 was used as the search model. A clear solution to the rotation/translation search was found, with a starting R-factor of 47.8% for data to 2.5 Å. The final model was built with a combination of automatic fitting in the program ArpWarp (http://www.arp-warp.org) and manual rebuilding on the graphics screen, using the program O Refinement in Refmac was carried out using all data in the resolution range 30.0-1.8 Å. Partial structure factors from a bulk-solvent model and anisotropic B-factor correction were supplied throughout the refinement. The R-factor for the current model is 0.22 (free R-factor, 7% of the data, 0.27). The refinement statistics are summarized in Table 5b.

The current model contains 307 out of 352 amino acid residues calculated on the basis of the construct. Interpretable electron density is seen for all residues from 454-760. Residues 442-453 at the N-terminus and 761 to 774 have not been modelled. In addition, the model contains one Zn²⁺ ion, one Mg²⁺ ion, one molecule of the inhibitor, the compound of Formula 1 (6,7-dimethoxy-4-[8-(4-methyl-piperazine-1-sulfonyl)-3,4-dihydro-1H-isoquinolin-2-yl]-quinazoline), and 312 water molecules.

A schematic representation of 6,7-dimethoxy-4-[8-(4-methyl-piperazine-1-sulfonyl)-3,4-dihydro-1H-isoquinolin-2-yl]-quinazoline is given below:

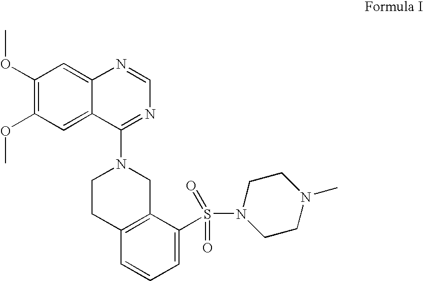

6,7-dimethoxy-4-[8-(4-methyl-piperazine-1-sulfonyl)-3,4-dihydro-1H-isoquinolin-2-yl]-quinazoline. Mass spectrum m/e calcd. for M+H=484.6. Found 484.2.

The compound of Formula 1 is disclosed in pending United States Provisional Application filed on Feb. 18, 2004 entitled “Tetrahydroisoquinolinyl Derivatives Of Quinazoline And Isoquinoline” and is hereby expressly incorporated by reference in its entirety.

TABLE 5a
Data statistics

| Resolution range | 30.0-1.8 Å |
|---|---|
| Number of observations | Total 112,444 |
| | Unique 37,865 |
| Completeness(%) | 91.4(53.9)¹ |
| l/s(I) | 12.2(0.8)¹ |
| R_{sym} | 0.063(0.67)^{1,2} |

¹Numbers in parentheses refer to the highest resolution range (1.80-1.86 Å)
²R_{sym} = Σ(I − <l>)/Σ<l>

TABLE 5b
Refinement statistics

| | |
|---|---|
| Nr. of reflections used | 34,917 |
| Nr. of reflections used for | 2,905 |
| R_{free} | |
| R_{cryst}/R_{free} | 0.227/0.273³ |
| Number of atoms | 2,839 |

³R = Σ||F_{obs}| − k|F_{calc}||/Σ|F_{obs}|

1. A phosphodiesterase 10A (PDE10A) crystal.

2. The PDE10A crystal of claim 1 which is derived from a mammal.

3. The PDE10A crystal of claim 2 wherein the mammal is a rat.

4. A crystal of the catalytic domain of PDE10A.

5. The crystal of claim 4 having a space group of R3 so as to form a unit cell of dimensions of about a=b=120.56 Å, and c=82.23 Å.

6. The crystal of claim 4, wherein said catalytic domain has a three dimensional structure characterized by the atomic structure coordinates of FIG. 4.

7. A PDE10A crystal according to claim 1 further comprising SEQ ID NO:2, or a homologue, analogue or variant thereof.

8. A crystal of a PDE10A/PDE10A ligand complex.

9. The crystal complex of claim 8 wherein the ligand is an antagonist or an inhibitor.

10. A crystal complex comprising a polypeptide with an amino acid sequence spanning amino acids Thr442 to Asp774 listed in SEQ ID NO:1, or a homologue, analogue or variant thereof.

11. The crystal complex of claim 10, wherein the homologue or variant has an amino acid identity of at least 98%, 95% or 90% with a polypeptide having an amino acid sequence spanning amino acids Thr442 to Asp774 listed in SEQ ID NO:1.

12. The crystal complex of claim 11, wherein the crystal comprises the atomic coordinates listed in FIG. 4.

13. The crystal complex of claim 10, wherein the homologue or variant thereof has a protein backbone comprising the atomic coordinates, or portions thereof, that are within a root mean square of +/−1.5, 1.2, 1.0, 0.7, 0.5, or even 0.2 Å of the atomic coordinates, or portions thereof, listed in FIG. 4.

14. A polypeptide comprising the amino acid sequence set forth in SEQ ID NO: 1 or a homologue, or variant thereof, wherein the molecules are arranged in a crystalline manner in a space group of R3 so as to form a unit cell of dimensions a=b=120.56 Å, and c=82.23 Å, and which effectively diffracts X-rays for determination of the atomic coordinates of PDE10A polypeptide to a resolution of about 1.8 Å.

15. A crystal of a protein-ligand molecule or molecular complex according to claim 10 comprising: (a) a polypeptide with an amino acid sequence from Thr442 to Asp774 listed in SEQ ID NO:1, or a homologue, or variant thereof; (b) a ligand; (c) wherein the crystal effectively diffracts X-rays for the determination of atomic coordinates of the protein-ligand complex to a resolution of greater than 1.8 Angstroms.

16. The crystal of claim 15 having a space group of R3 so as to form a unit cell of dimensions a=b=120.56 Å, and c=82.23 Å.

17. The crystal of claim 15 having a three-dimensional structure characterized by the atomic coordinates of FIG. 4.

18. The PDE10A crystal of claim 1 having the atomic coordinates set out in FIG. 4.

19. A method for generating the 3-D atomic coordinates of protein homologues of PDE10A using the X-ray coordinates of PDE10A described in FIG. 4, comprising: identifying the sequences of one or more proteins which are homologues of PDE10A; aligning the homologue sequences with the sequence of PDE10A (SEQ ID NO: 1); identifying structurally conserved and structurally variable regions between the homologue sequences, and PDE10A (SEQ ID NO:1); generating 3-D coordinates for structurally conserved residues, variable regions and side-chains of the homologue sequences from those of PDE10A; and combining the 3-D coordinates of the conserved residues, variable regions and side-chain conformations to generate a full or partial 3-D coordinates for said homologue sequences.

20. A method for identifying potential ligands for PDE10A, or homologues, analogues or variants thereof, comprising: displaying three dimensional structure of PDE10A enzyme, or portions thereof, as defined by atomic coordinates in FIG. 4, on a computer display screen; optionally replacing one or more PDE10A enzyme amino acid residues listed in SEQ ID NO:1, or one or more of the amino acids listed in Tables 1-4, or one or more amino acid residues selected from Leu625, Phe629, Val668, Phe686, Met703, Gln716 and Phe719, in said three-dimensional structure with a different naturally occurring amino acid or an unnatural amino acid; employing said three-dimensional structure to design or select said ligand; contacting said ligand with PDE10A, or variant thereof, in the presence of one or more substrates; and measuring the ability of said ligand to modulate the activity PDE10A.
